(19) United States
(12) Patent Application Publication
(10) Pub. No.: US 2024/0374630 A1
(43) Pub. Date: Nov. 14, 2024

(54) COMPOSITION FOR CAPTURING LEAD AND/OR CADMIUM IN A DIALYSIS FLUID

(71) Applicants: MEXBRAIN, Villeurbanne (FR); UNIVERSITE CLAUDE BERNARD LYON 1, Villeurbanne Cedex (FR); CENTRE NATIONAL DE LA RECHERCHE SCIENTIFIQUE - CNRS -, Paris (FR)

(72) Inventors: Olivier TILLEMENT, Fontaines Saint-Martin (FR); François LUX, Lyon (FR); Thomas BRICHART, Villeurbanne (FR); Coralie GRANGE, Villeurbanne (FR); Marco NATUZZI, Lyon (FR); Jordyn HOWARD, Lyon (FR); Juan SALAZAR ARIZA, Villeurbanne (FR); Aymeric COUTURIER, Paris (FR); Fabien ROSSETTI, Villeurbanne (FR)

(21) Appl. No.: 18/291,693

(22) PCT Filed: Jul. 28, 2022
(86) PCT No.: PCT/FR2022/051518

§ 371 Date: Jan. 24, 2024

(30) Foreign Application Priority Data
Jul. 30, 2021 (FR) FR2108374

(51) Int. Cl. A61K 31/715; A61K 31/395; A61M 1/16; A61P 39/04

(52) U.S. Cl. CPC A61K 31/715; A61K 31/395; A61M 1/1654; A61P 39/04

(57) ABSTRACT

The invention relates to a method for treating dialysis water and fluids, in particular for capturing certain toxic metals such as lead, aluminium, chromium, nickel or cadmium, and preventing their passage into the blood of a patient during dialysis, i.e. hemodialysis, hemofiltration or hemodiafiltration. In particular, it relates to a dialysis composition, comprising a polymer with a weight-average molecular weight of 20 kDa and 1000 kDa having at least one chelating agent of at least one toxic exogenous metal such as lead, aluminium, chromium, nickel and cadmium, said polymer being present in a sufficient quantity to be at a concentration comprised between 0.01 mg/L and 10 mg/L when used during a dialysis of a patient.

### TECHNICAL FIELD

The present disclosure relates to the field of dialysis techniques. The invention relates more particularly to methods for treating dialysis water and fluids, in particular for capturing certain toxic metals such as lead, aluminum, chromium, nickel or cadmium, thus reducing the concentration of metal cations that are liable to diffuse through a membrane, and preventing their passage into the blood during the dialysis of a patient, for example hemodialysis, hemofiltration or hemodiafiltration.

### PRIOR ART

Maintaining the homeostasis of the body's internal environment, i.e. all the body's biological fluids, is essential for its proper functioning. In many pathologies, systemic or local deregulations of metal homeostasis have been demonstrated.

In the body, metals are subject to numerous equilibria and are associated with complex functions. Certain metals, such as copper and iron, are essential for the functioning of the human body, while others have no recognized function and are associated with high toxicity. This is notably the case for heavy metals such as lead, mercury or cadmium. These metals may notably induce oxidative stress or interact with and modify the functions of essential biomolecules.

Cadmium and lead are thus mentioned by the World Health Organization (WHO) as two of the ten pollutants of major importance to human health (S. Satarug et al., Toxics, 2020). They are predominantly concentrated in the bones and kidneys, but their toxic effect is not limited to those organs, notably by inducing oxidative stress or DNA damage, by taking the place of other metals or by diverting certain proteins from their intended use, notably those containing sulfur compounds. Safety agencies have thus drastically lowered the acceptable levels of these compounds, although it has been shown that even a low content below these values is acknowledged as being toxic, and that any further reduction in this level would be beneficial. For lead, the WHO thus recommends a blood content of less than 100 μg·L⁻¹ in the case of adults, and the US agency recommends a content of 50 μg·L⁻¹ in the case of children (L. A. Alli, Interdiscip. Toxicol., 2015). For cadmium, the level is much lower, ranging from 0.3 to 1.2 μg·L⁻¹ for the WHO. Increasing the cadmium or lead content in the blood is thus notably linked to an increased risk of kidney disease, cardiovascular disease or cancer.

Hemodialysis and hemodiafiltration are currently the most conventionally used treatments for patients suffering from terminal-phase renal disease, and are unfortunately associated with excess mortality from infection or cardiovascular disease (M. Tonelli et al., BMC Medicine, 2009). These techniques consist in allowing, through a membrane permeable to small and medium molecular weight molecules, free exchange of ions and toxins between a fluid called a dialyzate and the patient's blood. This technique allows toxic molecules or atoms such as heavy metals to be extracted from the blood. A dialysis fluid consists essentially of water. Since the object of dialysis is to purify the blood, it goes without saying that the water used to prepare a dialysis fluid must be sterile and as pure as possible. Numerous studies have shown that long-term dialysis patients may be deficient in essential elements such as zinc and selenium, and over-concentrated in heavy metals such as lead and cadmium (A. Almeida et al., J. Trace Elem. Med. Biol., 2020). This is because patients undergoing hemodialysis are subjected to a large amount of dialyzate (>300 L/week), and thus even a trace amount of pollution in the dialyzate may lead to an overconcentration in heavy metals and proven clinical toxicity (G. Filler et al., Pediatr. Nephrol., 2014). Cadmium is, for example, associated with renal dysfunction (C. G. Sotomayor, Kidney Int., 2020), osteoporosis (K. A. James, Int. J. of Public Health, 2013) and arteriosclerosis (B. Messner, Arteriosclerosis, thrombosis and Vascular Bio., 2009) with increased cardiovascular mortality in patients exposed to high concentrations (S. Larsson, Int. J. of Epidemiology, 2016). These risks are all the greater in the case of children, for whom heavy metal toxicity is recognized as even more critical notably for neurocognitive development and growth. These risks are also elevated in the case of pregnant women for the development of the fetus.

The recommended purity of dialysis water is already quite strict, and is often of the order of ppb in maximum lead or cadmium concentration, i.e. much lower than the total concentration in the blood. Nevertheless, these concentrations do not appear to be low enough yet to avoid health problems linked to heavy metal overload. Specifically, while concentrations of heavy metals in the blood are very often higher than in the dialysis fluids used, it should be noted that in the blood, the vast majority of these metals are associated with cells (in particular erythrocytes) and only a few percent are found in plasma. The plasma concentration of lead and cadmium is thus fairly close to the initial heavy metal content in dialysis fluids. Even more precisely, of the metal content in plasma, a large majority is associated with large proteins (such as albumin) and thus may not diffuse through conventional dialysis membranes. The plasma concentration of heavy metal cations, Pb²⁺ and Cd²⁺, either free or associated with small species that can diffuse through a dialysis membrane, is thus much lower and often appears below the contents of the dialysis fluid. There is thus a risk of heavy metal contamination of the patient during dialysis, even if the dialysis fluid contents are below 1 ppb.

A dialysis fluid partially purified of free metals, and in particular of heavy metals such as lead or cadmium, is beneficial to dialysis treatment and particularly to hemodiafiltration, when the fluid is reinjected directly into the patient, upstream or downstream of dialysis. Heavy metals are known to promote oxidative stress, and a reduction in their content in dialysis fluids would be beneficial in terms of reducing the risk of cardiovascular disease, atherosclerosis, reduced kidney function, joint and bone problems, and also neurodegenerative diseases. Capturing heavy metals offers a means of safeguarding patients' endothelium.

It is extremely difficult to ensure the absence of metallic impurities in chemical reagents, or even in the water source required for formulating a dialysis fluid. This fact is even more problematic in emerging countries, making it necessary to implement technological solutions to ensure that dialysis does not contribute to an over-concentration of toxic metal species. In particular, the purification of the water used for preparing a dialysis fluid involves a long and expensive process requiring highly technical equipment. Moreover, the contents of elements that are potentially pollutant to the dialysis patient range from one location to another.

Thus, in general, in hemodialysis devices, the use of ultra-pure water, and also ultra-pure dialysis fluids, is widely recommended.

In particular, new devices such as ultrafiltration filters allow the content of endotoxins to be reduced directly on the dialysis fluid circulation line, before passage through the dialyzer, or even before direct reinsertion into the patient in the case of hemodiafiltration. Several tens of liters of dialysis fluid may thus be injected directly into the patient during a session, and the quality of the fluid is very important. While these devices, for instance the DIASAFE Plus hemodialysis filter, allow effective retention of microbial contamination and/or bacterial endotoxins, they have no efficacy when it comes to retaining free metals.

Thus, it would be beneficial to propose a solution which at least partially dispenses with these devices and the place where the dialysis fluid is prepared, in order to prevent pollution of the dialysis patient.

It is in this context that the present invention relates notably to the use of a chelating polymeric adjuvant, which is present in very small amounts in the dialyzate and which can chelate undesirable heavy metals, while at the same time being of sufficient size to prevent its release into the body during dialysis.

One object of the invention is thus to propose a method for preparing a composition for dialysis, independent of the place where the dialysis is performed.

Another object of the invention is to propose a dialysis composition which has extremely low levels of diffusable heavy metals (lead and cadmium in the form of free cations or cations associated with small molecules/complexing ions), below the ppb level and preferably below 0.1 ppb.

An additional object is to propose a method for preventing pollution of the dialysis patient with exogenous metals such as lead or cadmium.

These and other objects are achieved by means of the composition according to the invention and its uses detailed below.

### SUMMARY

The present invention relates to a dialysis composition comprising a polymer with a weight-average molecular mass of between 20 kDa and 1000 kDa bearing at least one agent for chelating at least one metal such as lead, aluminum, chromium, nickel or cadmium, said polymer being present in an amount sufficient to be in a concentration of between 0.01 mg/L and 10 mg/L in the dialyzate during the dialysis of a patient.

The present invention also relates to a method for capturing at least one toxic metal, such as lead, aluminum, chromium, nickel or cadmium, present in a dialysis fluid, comprising a step of adding to said dialysis fluid a sufficient amount of a polymer with a weight-average molecular mass of between 20 kDa and 1000 kDa bearing at least one agent for chelating at least one toxic metal such as lead, aluminum, chromium, nickel or cadmium, thus allowing said toxic metal to be captured by chelation with the chelating agent of said polymer.

The invention also relates to the use of a polymer with a weight-average molecular mass of between 20 kDa and 1000 kDa bearing at least one agent for chelating at least one toxic metal such as lead, aluminum, chromium, nickel or cadmium, to prepare a composition for dialysis, for example for hemodialysis, hemodiafiltration or hemofiltration, in particular a composition according to the invention.

The features outlined in the following paragraphs may optionally be implemented independently of each other or in combination with each other.

### BRIEF DESCRIPTION OF THE DRAWINGS

Other features, details and advantages will appear on reading the detailed description below, and on analyzing the attached drawings, in which:

FIG. 1 is a scheme describing the principle of Vivaspin ultrafiltration.

FIG. 2 is a graph representing the efficiency of Pb²⁺ and Cd²⁺ capture according to Example 1.

FIG. 2a is an HPLC-MS chromatogram of the detection of Pb²⁺ interacting with the Mex-CD1 polymer according to Example 1.

FIG. 2b is an HPLC-MS chromatogram of the detection of Cd²⁺ interacting with the Mex-CD1 polymer according to Example 1.

FIG. 3 is an HPLC-MS chromatogram of the detection of Pb²⁺ in acetic acid according to Example 2.

FIG. 4 is an HPLC-MS chromatogram of the detection of Pb²⁺ in citric acid according to Example 2.

FIG. 5 is an HPLC-MS chromatogram of the detection of Pb²⁺ in acetic acid according to Example 3.

FIG. 6 is an HPLC-MS chromatogram of the detection of Pb²⁺ in citric acid according to Example 3.

FIG. 7 is a graph representing the efficiency of Pb²⁺ capture according to Example 4.

FIG. 8 represents two graphs showing the efficiency of Pb²⁺ and Cd²⁺ capture, at high and low concentration, according to Example 5.

FIG. 9 is a graph representing the efficiency of Cu²⁺ and Mn²⁺ capture, according to Example 5.

FIG. 10 is a graph representing the efficiency of Pb²⁺ capture, according to Example 6.

FIG. 11 is a graph representing the efficiency of Cd²⁺, Cu²⁺ and Pb²⁺ capture, according to Example 7.

FIG. 12 is a graph representing the efficiency of Cd²⁺, Cu²⁺ and Pb²⁺ capture, according to Example 8.

FIG. 13 is a graph representing the efficiency of Cd²⁺, Cu²⁺ and Pb²⁺ capture, according to Example 9.

FIG. 14 is a graph representing the efficiency of Cd²⁺, Cu²⁺ and Pb²⁺ capture, according to Example 10.

FIG. 15 represents a chromatogram representing the stability of MEX-CD1 in citric acid according to Example 11.

FIG. 16 represents a chromatogram representing the stability of PEG@DOTAGA in citric acid according to Example 11.

FIG. 17 is a chromatogram representing the chelation of Pb²⁺ and Cd²⁺ according to Example 12.

FIG. 18 is a chromatogram representing the chelation of Pb²⁺ and Cd²⁺ in acid medium according to Example 12.

FIG. 19 is a chromatogram representing the chelation of aluminum according to Example 13.

FIG. 20 is a chromatogram representing the chelation of aluminum in the presence of citrate according to Example 13.

FIG. 21 is a graph representing the aluminum concentration measured by ICP-MS according to Example 14.

FIG. 22 is a graph representing the percentage of aluminum extracted according to Example 14.

### DETAILED DESCRIPTION

The invention relates to a dialysis composition, said composition comprising a polymer with a weight-average molecular mass of between 20 kDa and 1000 kDa and bearing at least one agent for chelating at least one metal such as lead, aluminum, chromium, nickel or cadmium, said polymer being in an amount sufficient to be in a concentration of between 0.01 mg/L and 10 mg/L in the dialyzate (or dialysis fluid) during the dialysis of a patient.

For the purposes of the invention, the term “dialysis” encompasses processes for purifying blood in patients who require it, for example patients suffering from acute or chronic renal failure, notably terminal chronic renal failure, and comprising the use of a dialyzer. The dialyzer comprises a porous or semi-permeable membrane, allowing the passage of small molecules by diffusion or convection, while at the same time blocking the passage of larger molecules, for example proteins. The membrane thus has a “cut-off” threshold, i.e. the molecular mass above which the membrane is considered strictly impermeable, i.e. capable of blocking 90%, 95%, 96%, 97%, 98%, 99%, 99.5%, 99.6%, 99.7%, 99.8%, or even up to at least 99.9% of molecules with a molecular mass greater than the cut-off threshold.

The term “dialysis” or “extra-renal purification” in particular covers hemodialysis, hemofiltration and hemodiafiltration. Dialysis denotes all the extrarenal purification methods allowing uremic toxins to be purified and the hydroelectrolytic disorders resulting from renal dysfunction (potassium, calcium, phosphorus, acid, base) to be corrected. In practice, the cut-off point of a semi-permeable membrane for hemodialysis is less than 20 to 50 kDa for high-permeability membranes. In the hemodialysis context, exchanges across the porous or semi-permeable membrane take place mainly via a concentration gradient on either side of the membrane, the mechanism known as diffusion. In the context of hemofiltration, exchanges take place via a pressure gradient, from the dialyzate to the blood, known as convection. Hemodiafiltration is the most commonly prescribed technique, combining diffusion and convection (B. Canaud, Principes et modalités d'application de l'hémodialyse au traitement de l'insuffisance rénale chronique. Nephrology and Therapeutics 2009).

The extrarenal purification devices used with the present invention thus allow exchanges between dialyzate and blood through a semi-permeable membrane, and comprise (1) a tank containing ultra-pure water, (2) a generator which ensures extracorporeal blood circulation, the circulation of the dialyzate and generates the dialyzate (or dialysis fluid), (3) a semi-permeable membrane, also known as a dialyzer, (4) an extracorporeal blood circulation device and (5) a vascular access.

The term “extracorporeal blood circulation device” typically means a device that allows venous blood flow to be diverted into a circuit outside the body, with a circulation flow rate of 300 to 400 ml/min in adults (variable in children, in neonatology >10 ml/min).

The term “dialyzate” or “dialysis fluid” (used interchangeably) refers to the fluid prepared by the generator, and used during the dialysis session to correct hydroelectrolytic disorders. The dialyzate is a bath mixture of acid (acetic acid, citric acid or hydrochloric acid), ions (potassium, sodium, chlorine, calcium, magnesium), glucose, sterile ultrapure water and a bicarbonate buffer. When using an ultrafiltration purification device (e.g. DIASAFE® Plus or DIACLEAR® Ultrafiltrate), the dialyzate composition may be measured upstream or downstream of an ultrapurification filter. It is preferentially intended to be measured upstream of these filters, at the inlet connection. Since the dialyzate flow rate is generally 500 to 700 ml/min, and dialysis sessions last 4 hours, a dialyzate generator produces continuously and extemporaneously 120 to 170 liters of dialyzate per session. Certain short hemodialysis protocols (2 hours per session, and 6 sessions per week), known as daily hemodialysis, use generators (NxStage, Physidia S3) that require dialyzate in 5-liter sterile bags, of the same composition as described previously.

The term “dialysis composition” thus refers to any composition that is suitable for use in the preparation of a dialyzate or dialysis fluid. Thus, for the purposes of the invention, the term “dialysis composition” encompasses the dialyzate, or a concentrated solution (additive) allowing the preparation of the dialysis fluid after dilution in sterile water, and, where appropriate with other additives. Typically, in one embodiment, the dialysis composition according to the invention is a concentrated dialysis solution or a concentrated-solution additive. Said concentrated solutions notably comprise acidic concentrated solutions, comprising at least a sufficient amount of acid, for example chosen from acetic acid, hydrochloric acid or citric acid, combined with a mixture of ions, such as potassium, sodium, chlorine, calcium or magnesium, with glucose, to which solution is added bicarbonate buffer and sterile ultrapure water.

In another embodiment, the dialysis composition according to the invention is a concentrated solution also comprising one or more electrolytes, in particular chosen from sodium, potassium, chlorine, magnesium or calcium, and bicarbonates.

In one particular embodiment, the dialysis composition is a concentrated solution comprising, in addition to said polymer, an acid and several electrolytes, in particular chosen from sodium, potassium, chlorine, magnesium and calcium, and bicarbonates.

In another particular embodiment, the dialysis composition according to the invention is a concentrated solution packaged in a bag suitable for a dialysis device, preferably a bag containing a solution volume of between 500 and 5000 mL and an amount of said polymer of between 5 and 5000 mg and preferably between 10 and 100 mg.

### The Polymer

In accordance with the invention, the polymer has a weight-average molecular mass of between 20 kDa and 1000 kDa. Thus, when the dialysis composition is used in a dialysis device, the polymer has an average molecular mass above the cut-off threshold of the semi-permeable membrane used in the dialysis device. Preferably, the polymer has a weight-average molecular mass of between 100 kDa and 900 kDa, more preferably between 250 kDa and 750 kDa and even more preferably between 400 kDa and 600 kDa.

In a particular embodiment of the invention, the polymer is a random polysaccharide bearing at least one chelating agent.

The average molecular mass of the polymer may be determined by size exclusion chromatography.

Preferably, the polymer according to the invention, preferably the polysaccharide of formula I or II below, has a complexation constant of at least 10¹⁵ for a transition element d or f.

In particular, in an advantageous mode, the polymer is a random polysaccharide of formula I

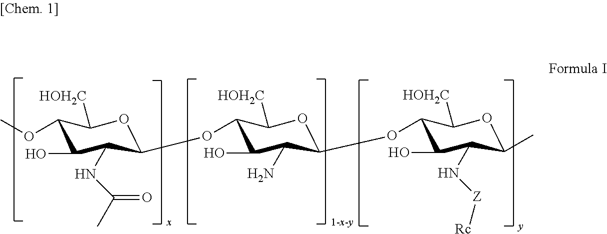

in which:
each Rc independently represents a group including a chelating agent,
each Z independently represents a linkage which may be a single bond or a hydrocarbon-based chain including between 1 and 12 carbon atoms, said chain possibly being linear or branched and possibly including one or more unsaturations and possibly including one or more heteroatoms, preferably chosen from nitrogen, oxygen, sulfur and atoms of the halogen family,
x is between 0.005 and 0.7, preferably between 0.05 and 0.7, preferentially between 0.2 and 0.6 and more preferentially between 0.25 and 0.4,
y is between 0.01 and 0.7, preferably between 0.05 and 0.2,
the ratio y/x is greater than or equal to 0.05, preferably greater than or equal to 0.15, and
the sum x+y is greater than or equal to 0.30, preferably greater than or equal to 0.35.

It is understood that, in formula I above, several groups Rc may be present in the polysaccharide. These groups Rc may be identical to or different from each other. They are all independently chosen from groups bearing a chelating agent. The same applies to the linkages Z: several linkages Z may be present, and they may be identical to or different from each other.

According to another embodiment, in formula I, x is between 0.05 and 0.6,
y is between 0.1 and 0.9,
the ratio y/x being greater than 0.16, and
the sum x+y being greater than 0.4.

According to one embodiment, the polysaccharide of formula I is a polysaccharide of formula II

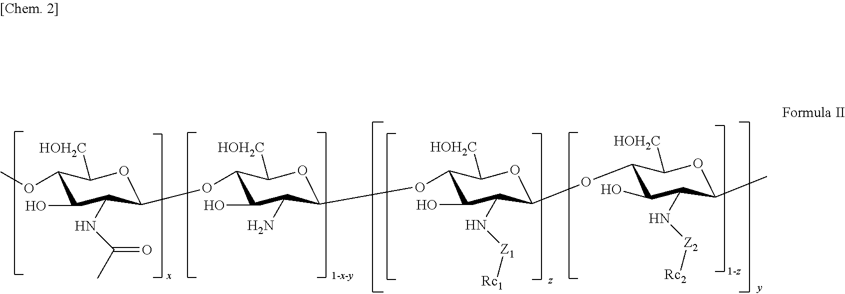

in which:
Rc₁ and Rc₂ are different, and are groups including a chelating agent,
Z₁ and Z₂, which may be identical or different, are linkages which may be a single bond or a hydrocarbon-based chain including between 1 and 12 carbon atoms, said chain possibly being linear or branched and possibly including one or more unsaturations and possibly including one or more heteroatoms, preferably chosen from nitrogen, oxygen, sulfur and atoms of the halogen family.
x is between 0.005 and 0.7, preferably between 0.05 and 0.7, preferentially between 0.2 and 0.6 and more preferentially between 0.25 and 0.4,
y is between 0.01 and 0.7, preferably between 0.05 and 0.2,
the ratio y/x being greater than or equal to 0.05, preferably greater than or equal to 0.15,
the sum x+y being greater than or equal to 0.30, preferably greater than or equal to 0.35, and
z is between 0.5 and 1.

In this specific embodiment, the polysaccharide of formula II may comprise:
only one type of group including a chelating agent, Rc₁, when z=1, or
two types of groups including a chelating agent, Rc₁ and Rc₂, when 0.5≤z<1.

According to one embodiment, z is between 0.8 and 0.99, and the group Rc₁ is thus largely predominant.

According to another embodiment, in formula II, x is between 0.05 and 0.6,
y is between 0.1 and 0.9,
the ratio y/x being greater than 0.16, and
the sum x+y being greater than 0.4.

The term “Rc-type group” means the groups Rc in the polysaccharide of formula I, and the groups Rc₁ and Rc₂, where the group Rc₂ is present, in the polysaccharide of formula II.

The group Rc is a chelating agent. In other words, the group Rc allows one or more metals to be chelated to form a complex.

In accordance with the invention, the groups Rc, Rc₁ and Rc₂ are chelating agents. In other words, the groups Rc, Rc₁ et Rc₂ allow one or more metals to be chelated to form a complex.

Each of the groups Rc, Rc₁ and Rc₂ may contain one or more coordination sites. Preferably, the coordination site is a nitrogen or oxygen atom. Advantageously, each of the groups Rc, Rc₁ and Rc₂ includes between 4 and 8 coordination sites, more advantageously between 6 and 8 coordination sites and even more advantageously each of the groups Rc, Rc₁ and Rc₂ includes 8 coordination sites.

The term “coordination site” means a single function that is capable of chelating a metal. For example, an amine function represents a coordination site by formation of a dative bond between the nitrogen atom and the metal, and a hydroxamic acid function also represents a coordination site by formation of a dative bond between the oxygen of the carbonyl unit and by a covalent bond with the oxygen of the N-oxide unit, the coordination site thus forming a five-membered ring.

In one embodiment of the invention, for the polysaccharide of formula I, each group Rc is independently chosen from the group consisting of DOTA (1,4,7,10-tetraazacyclododecane-N,N′,N″,N″-tetracetic acid), NOTA (1,4,7-triazacyclononane-1,4,7-triacetic acid), NODAGA (1,4,7-triazacyclononane-1-glutaric-4,7-diacetic acid), DOTAGA (2-(4,7,10-tris(carboxymethyl)-1,4,7,10-tetraazacyclododecan-1-yl) pentanedioic acid), DOTAM (1,4,7,10-tetrakis(carbamoylmethyl)-1,4,7,10-tetraazacyclododecane), DO3AM (2′4,7,10-tris(2-amino-2-oxoethyl)-1,4,7,10-tetraazacyclododecan-1-yl) acetic acid), NOTAM (1,4,7,10-tetrakis(carbamoylmethyl)-1,4,7-triazacyclononane), DOTP (1,4,7,10-tetraazacyclododecane 1,4,7,10-tetrakis(methylene phosphonate), NOTP (1,4,7,10-tetrakis(methylene phosphonate)-1,4,7-triazacyclononane), TETA (1,4,8,11-tetraazacyclotetradecane-N,N′,N″,N′″-tetracetic acid), TETAM (1,4,8,11-tetraazacyclotetradecane-N,N′,N″, N″-tetrakis(carbamoylmethyl), DTPA (diethylenetriaminepentaacetic acid) and DFO (deferoxamine), preferably from the group consisting of DOTAGA, DFO, DOTAM and DTPA and more preferably the group Rc is DOTAGA.

In one embodiment of the invention, for the polysaccharide of formula II, Rc₁ and Rc₂ are independently chosen from the group consisting of DOTA, NOTA, NODAGA, DOTAGA, DOTAM, DO3AM, NOTAM, DOTP, NOTP, TETA, TETAM, DTPA and DFO, preferably from the group consisting of DOTAGA, DFO, DOTAM and DTPA.

According to one embodiment, for the polysaccharide of formula II, the group Rc₁ is DOTAGA, and preferably z=1.

According to one embodiment, for the polysaccharide of formula II, the group Rc₁ is DOTAGA, and the group Rc₂ is DFO.

The term “Z-type linkage” means Z in the polysaccharide of formula I, and the linkages Z₁ and Z₂, when the linkage Z₂ is present, in the polysaccharide of formula II.

The choice of linkages Z, Z₁ and Z₂ in formulae I and II depends essentially on the groups Rc, Rc₁ and Rc₂ and the metal to be chelated. Specifically, notably for steric reasons, the groups Rc, Rc₁ and Rc₂ may be more or less close to the 6-membered nitrogen ring of the glucosamine unit.

Preferably, in formula I, each Z is independently a single bond or a hydrocarbon-based chain including between 1 and 12 carbon atoms, said chain possibly being linear or branched and possibly including one or more unsaturations and possibly including one or more heteroatoms, preferably chosen from nitrogen, oxygen, sulfur and atoms of the halogen family.

According to one embodiment, in formula I, each Z is independently selected from the group consisting of: a bond, a linear or branched alkyl chain including between 1 and 12 carbon atoms, and a linear or branched alkenyl chain including between 1 and 12 carbon atoms, said alkyl and alkenyl chains possibly being interrupted with one or more C₆-C₁₀ aryl groups, and/or with one or more heteroatoms or groups selected from the group consisting of —O—, —S—, —C(O)—, —NR′—, —C(O)NR′—, —NR′—C(O)—, —NR′—C(O)—NR′—, —NR′—C(O)—O—, —O—C(O)NR′, —C(S) NR′—, —NR′—C(S)—, —NR′—C(S)—NR′ said alkyl and alkenyl chains possibly being substituted with one or more groups selected from the group consisting of halogens, —OR′, —COOR′, —SR′, —NR′2, each R′ is independently H or a C₁-C₆ alkyl.

Advantageously, in formula I, each Z is independently selected from the group consisting of: a bond and a linear or branched alkyl chain including between 1 and 12 carbon atoms, said alkyl chain possibly being interrupted with one or more C₆-C₁₀ aryl groups, and/or with one or more heteroatoms or groups selected from the group consisting of —O—, —S—, —C(O)—, —NR′—, —C(O)NR′—, —NR′—C(O)—, —C(S) NR′—, —NR′—C(S)—, —NR′—C(S)—NR′, each R′ is independently H or C₁-C₆ alkyl.

In one particular embodiment, each Z is an alkyl chain including between 1 and 12 carbon atoms.

In another particular embodiment, each Z is a polyethylene glycol (PEG).

Preferably, in formula II, Z₁ and Z₂ are independently a single bond or a hydrocarbon-based chain including between 1 and 12 carbon atoms, said chain possibly being linear or branched and possibly including one or more unsaturations and possibly including one or more heteroatoms, preferably chosen from nitrogen, oxygen, sulfur and atoms of the halogen family.

According to one embodiment, in formula II, Z₁ and Z₂ are independently selected from the group consisting of: a bond, a linear or branched alkyl chain including between 1 and 12 carbon atoms, and a linear or branched alkenyl chain including between 1 and 12 carbon atoms, said alkyl and alkenyl chains possibly being interrupted with one or more C₆-C₁₀ aryl groups, and/or with one or more heteroatoms or groups selected from the group consisting of —O—, —S—, —C(O)—, —NR′—, —C(O)NR′—, —NR′—C(O)—, —NR′—C(O)—NR′—, —NR′—C(O)—O—, —O—C(O)NR′, —C(S) NR′—, —NR′—C(S)—, —NR′—C(S)—NR′, said alkyl and alkenyl chains possibly being substituted with one or more groups selected from the group consisting of halogens, —OR′, —COOR′, —SR′, —NR′2, each R′ is independently H or a C₁-C₆ alkyl.

Advantageously, in formula II, Z₁ and Z₂ are independently selected from the group consisting of: a bond and a linear or branched alkyl chain including between 1 and 12 carbon atoms,
said alkyl chain possibly being interrupted with one or more C₆-C₁₀ aryl groups, and/or with one or more heteroatoms or groups selected from the group consisting of —O—, —S—, —C(O)—, —NR′—, —C(O)NR′—, —NR′—C(O)—, —C(S) NR′—, —NR′—C(S)—, —NR′—C(S)—NR′,
each R′ is independently H or a C₁-C₆ alkyl.

In one particular embodiment, Z₁ and/or Z₂ is an alkyl chain including between 1 and 12 carbon atoms.

In another particular embodiment, Z₁ and/or Z₂ is a polyethylene glycol (PEG). The polymer of formula I is composed of three monomer units, namely an N-acetylglucosamine type unit A, a glucosamine type unit B and a glucosamine type unit C functionalized with a chelating agent (Rc) linked by a linkage Z to the glucosamine nitrogen.

The polysaccharide according to the invention is composed of three monomer units, namely an N-acetylglucosamine type unit A, a glucosamine type unit B and a glucosamine type unit C functionalized with a chelating agent (Rc type) linked by a linkage (Z type) to the glucosamine nitrogen.

The polymer of formula I or II is a random polymer. In other words, the sequence of the various monomer units A, B and type C is random.

In formulae I and II, x represents the proportion of units A and x is between 0.05 and 0.7, preferably x is between 0.2 and 0.6, more preferably x is between 0.25 and 0.4.

In formulae I and II, y represents the proportion of units C and y is between 0.1 and 0.7, preferably between 0.05 and 0.2.

The ratio y/x is preferably between 0.15 and 1.5.

The sum x+y is preferably between 0.35 and 0.8.

The remaining monomer units in formulae I and II are units B. Thus, in formula I, the proportion of units B is equal to 1-x-y.

In another particular embodiment of the invention, the polymer is a biocompatible polymer, for example of the polyethylene glycol (PEG), poly(lactic-co-glycolic acid) (PLGA), polyacrylamide, polyamine or polycarboxylic type. In this case, the biocompatible polymer, preferably a PEG, comprises a chelating agent preferably selected from the group consisting of EDTA, DOTA, NOTA, NODAGA, DOTAGA, DOTAM, DO3AM, NOTAM, DOTP, NOTP, TETA, TETAM, DTPA and DFO, and more preferentially from the group consisting of DOTAGA, EDTA, DFO, DOTAM and DTPA.

In particular, the polymer is a PEG comprising a chelating agent which may be chosen from a PEG functionalized with DOTAGA, a PEG functionalized with EDTA, a PEG functionalized with DTPA, a PEG functionalized with DFO, and a PEG functionalized with DOTA.

According to one embodiment, the polymer is chosen from polysaccharides of formula I, polysaccharides of formula II and PEGs comprising a chelating agent.

The chelating agent of the polymer is capable of chelating heavy metals which may be present in the waters and liquids used for the preparation of the dialyzate. In particular, in a preferred embodiment, the concentration of the polymer in the dialysis composition is sufficient to allow a free residual heavy metal concentration in the dialyzate of less than 0.1 ppb. The term “free residual concentration” means the concentration of toxic heavy metals, typically lead and cadmium, which are not complexed to the polymer chelating agent in the dialyzate and which may therefore pass through the semi-permeable membrane of the dialysis device.

In general, according to the invention, the chelating agent is thus preferably present in the dialyzate during the step of dialysis of a patient at a concentration of between 10⁻⁴ mmol·L⁻¹ and 10⁻⁶ mmol·L⁻¹.

In a preferred embodiment, in particular in combination with the advantageous embodiment in which the polymer is of formula I above, the dialysis composition comprises a sufficient amount of polymer to be in a concentration in the dialyzate of between 0.1 mg/L and 10 mg/L, where appropriate after dilution. Thus, for example, in the case of a concentrated solution to be diluted 45-fold, the concentration of polymer in the concentrated solution is preferably between 4.5 and 450 mg/L.

Advantageously, the polymer used is stable in acid, in particular acetic acid or citric acid.

In one embodiment, the present invention is directed toward an additive, in particular a ready-to-use concentrated solution for dialysis, for example hemodialysis, comprising
(i) a polymer with a weight-average molecular mass of between 20 kDa and 1000 kDa, bearing at least one agent for chelating at least one toxic exogenous metal such as lead, aluminum, chromium, nickel or cadmium, in particular a polymer of formula I or II, or a PEG comprising a chelating agent, preferably in a concentration of between 4.5 and 450 mg/L;
(ii) where appropriate, (a) one or more electrolytes such as potassium, sodium, chlorine, calcium, magnesium, bicarbonate, acetate or citrate, and (b) at least one sugar, such as glucose or dextrose.

Said concentrated solution may be packaged in bag, cartridge or canister form, for example for 1/45 or 1/35 dilution with a dialyzate generator.

In one embodiment, the present invention is directed toward an acidic concentrated solution, in particular a ready-to-use acidic concentrated solution for dialysis, for example hemodialysis, comprising
(i) an acid chosen from citric acid, acetic acid or hydrochloric acid,
(ii) a polymer with a weight-average molecular mass of between 20 kDa and 1000 kDa, bearing at least one agent for chelating at least one toxic exogenous metal such as lead, aluminum, chromium, nickel or cadmium, in particular a polymer of formula I or II, or a PEG comprising a chelating agent, preferably in a concentration of between 4.5 and 450 mg/L;
(iii) where appropriate, (a) one or more electrolytes such as potassium, sodium, chlorine, calcium, magnesium, bicarbonate, acetate or citrate, and (b) at least one sugar, such as glucose or dextrose.

Said acidic concentrated solution may be packaged in bag, cartridge or canister form, for example for 1/45 or 1/35 dilution with a dialyzate generator.

The invention also relates to a PEG with a weight-average molecular mass of between 20 kDa and 1000 kDa bearing at least one agent for chelating at least one metal such as lead, aluminum, chromium, nickel or cadmium, the chelating agent preferably being selected from the group consisting of EDTA, DOTA, NOTA, NODAGA, DOTAGA, DOTAM, DO3AM, NOTAM, DOTP, NOTP, TETA, TETAM, DTPA and DFO, and more preferentially from the group consisting of DOTAGA, EDTA, DFO, DOTAM and DTPA. Advantageously, the PEG comprises between 1 μmol and 1 mmol of chelating agent per gram of polymer.

The invention also relates to a dialysis composition comprising such a PEG, said PEG being present in an amount sufficient to be in a concentration of between 0.01 mg/L and 10 mg/L in the dialyzate during the dialysis of a patient.

### Capture Method

The compositions described above are advantageously used for capturing toxic metals which may be present in the waters and liquids used for preparing the dialyzate.

Consequently, according to another aspect, the invention relates to a method for capturing at least one toxic metal, such as lead, aluminum, chromium, nickel or cadmium, which may be present in a dialysis fluid, comprising a step of adding to said dialysis fluid a sufficient amount of a polymer with a weight-average molecular mass of between 20 kDa and 1000 kDa bearing at least one agent for chelating at least one toxic exogenous metal, such as lead, aluminum, chromium, nickel or cadmium, and notably one of the polymers described in the preceding paragraph, more preferentially the polymer of formula (I) or (II), or a PEG comprising a chelating agent.

In one embodiment, the polymer is present in a concentrated solution suitable for use in a dialysis device, as described above, for example an acidic concentrated solution.

In one embodiment, the dialysis fluid comprising the polymer is introduced into the tank of the dialysis device which may be connected to an extracorporeal blood circulation device comprising a porous dialysis membrane, typically having a cut-off threshold below the average molecular mass of said polymer, for example less than 20 kDa. The polymer captures any toxic metals that may be present in the water or liquids or powders used in the preparation of the dialysis fluid by complexation with the chelating agent. The polymer does not pass into the bloodstream due to its molecular mass, which is greater than the cut-off threshold of the semi-porous membrane of the dialysis device.

In another embodiment, the dialysis fluid comprising the polymer is introduced upstream of an ultrafiltration filter, and in particular upstream of a filter which allows an ultra-pure dialysis fluid to be obtained, in particular with a microbial contamination level <0.1 CFU/ml and/or a bacterial endotoxin level <0.03 IU/ml. Thus, in addition to conventional endotoxin capture, the dialysis fluid comprising the polymer allows upstream toxic metals to be sequestered, thus purifying the dialysis water to less than 1 ppb of heavy metals and even less than 0.1 ppb of lead and/or cadmium in particular.

Thus, for example, the dialysis fluid comprising the polymer may be injected upstream of a Diasafe® Plus or DIACLEAR® Ultrafiltrate type filter or equivalent, it may also be injected downstream, directly before the dialyzer in contact with the patient's blood.

In a particular embodiment, the capture method according to the present invention is used for capturing aluminum, for example so as to obtain an uncomplexed aluminum concentration of less than 1 ppb in the dialyzate.

In a particular embodiment, the capture method according to the present invention is used for capturing lead, for example so as to obtain an uncomplexed lead concentration of less than 0.1 ppb in the dialyzate, preferably in the dialyzate at the dialyzer inlet and/or for direct injection into the blood, i.e. upstream or downstream of the filter.

In a particular embodiment, the capture method according to the present invention is used for capturing cadmium, for example so as to obtain an uncomplexed cadmium concentration of less than 0.01 ppb in the dialyzate, preferably in the dialyzate at the dialyzer inlet and/or for direct injection into the blood, i.e. upstream or downstream of the filter.

In one embodiment, the present method is performed in a dialysis session of a patient, for example a patient suffering from acute or chronic renal failure. In one embodiment, at least 120 L of dialysis fluid may be used.

The invention also relates to a method for treating liquids used in the preparation of a dialysis fluid, said method comprising a step of adding to said dialysis fluid a sufficient amount of a polymer with a weight-average molecular mass of between 20 kDa and 1000 kDa bearing at least one agent for chelating at least one toxic exogenous metal such as lead, aluminum, chromium, nickel or cadmium, and notably one of the polymers described in the preceding paragraph, more preferentially the polymer of formula (I).

### Use of the Polymer

According to a third aspect, the invention relates to the use of a polymer with a weight-average molecular mass of between 20 kDa and 1000 kDa bearing at least one agent for chelating at least one toxic exogenous metal such as lead, aluminum, chromium, nickel or cadmium, in particular a polymer as defined above, and notably the polymer of formula (I), to prepare a dialysis fluid, an acidic concentrated solution, or a powdered bicarbonate concentrate or any other solution containing electrolytes.

The invention also relates to the process for preparing a dialysis fluid, said process comprising the use of a dialysis fluid composition as described above, and comprising a polymer with a weight-average molecular mass of between 20 kDa and 1000 kDa bearing at least one agent for chelating at least one toxic metal such as lead, aluminum, chromium, nickel or cadmium, in a dialyzate generator so as to produce a dialyzate comprising said polymer in a concentration sufficient to complex said toxic metals.

The invention is also directed toward liquid concentrates, for example packaged in the form of a bag, cartridge or canister suitable for use in a dialysis device, and comprising a polymer with a weight-average molecular mass of between 20 kDa and 1000 kDa bearing at least one agent for chelating at least one toxic exogenous metal such as lead, aluminum, chromium, nickel or cadmium, notably a polymer as defined above, and in particular the polymer of formula (I);

for use in a dialysis process in a patient in need thereof, for example a patient suffering from acute or chronic renal failure. Such liquid concentrates may also comprise an acid, for example citric, acetic or hydrochloric acid.

The polymer is in an amount sufficient to allow the capture of toxic metals which may be present in the liquids used in the preparation of the dialyzate.

### Use of the Dialysis Composition

Advantageously, the dialysis composition according to the invention may be used on patients with plasma lead contents above 1 ppb or plasma cadmium contents above 0.1 ppb.

Advantageously, the dialysis composition according to the invention may be used on patients also presenting with cardiovascular disease and/or neurodegenerative disease and/or joint problems or brittle bones.

Advantageously, the dialysis composition according to the invention may be used on patients who are due to undergo a kidney transplant within the year and preferably within 3 months.

### Implementation Variants

According to a variant 1, the invention relates to a dialysis composition, comprising a polymer with a weight-average molecular mass of between 20 kDa and 1000 kDa bearing at least one agent for chelating at least one metal such as lead, aluminum, chromium, nickel or cadmium, said polymer being present in an amount sufficient to be in a concentration of between 0.01 mg/L and 10 mg/L in the dialyzate during the dialysis of a patient.

Variant 2: Dialysis composition according to variant 1, characterized in that the metal is chosen from heavy metals such as lead or cadmium.

Variant 3: Dialysis composition according to variant 1 or 2, characterized in that the chelating agent is chosen from the group consisting of DOTA, NOTA, NODAGA, DOTAGA, DOTAM, DO3AM, NOTAM, DOTP, NOTP, TETA, TETAM, DTPA, EDTA and DFO, preferably from the group consisting of DOTAGA, DFO, DOTAM and DTPA and more preferably DOTAGA.

Variant 4: Dialysis composition according to one of variants 1 to 3, characterized in that the polymer is a random polysaccharide bearing at least one chelating agent.

Variant 5: Dialysis composition according to one of variants 1 to 4, characterized in that the polymer is a random polysaccharide of formula I:

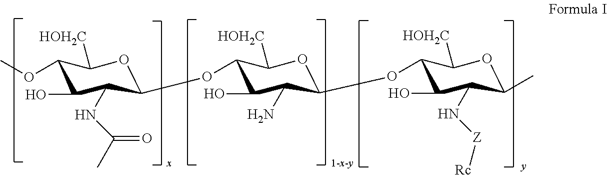

in which:
each Rc independently represents a group including a chelating agent,
each Z independently represents a linkage which may be a single bond or a hydrocarbon-based chain including between 1 and 12 carbon atoms, said chain possibly being linear or branched and including one or more unsaturations and possibly including one or more heteroatoms, preferably chosen from nitrogen, oxygen, sulfur and atoms of the halogen family, x is between 0.005 and 0.7, preferably between 0.05 and 0.7, preferentially between 0.2 and 0.6, and more preferentially between 0.25 and 0.4,
y is between 0.01 and 0.7, preferably between 0.05 and 0.2,
the ratio y/x is greater than or equal to 0.05, preferably greater than or equal to 0.15, and
the sum x+y is greater than or equal to 0.30, preferably greater than or equal to 0.35.

Variant 6: Dialysis composition according to one of variants 1 to 4, characterized in that the polymer is a biocompatible polymer, for example of the polyethylene glycol (PEG) type.

Variant 7: Dialysis composition according to one of variants 1 to 6, characterized in that it is a concentrated solution or an additive for a concentrated dialysis solution, preferably an acidic concentrated solution comprising, in addition to the polymer, a sufficient amount of acetic acid, hydrochloric acid or citric acid.

Variant 8: Dialysis composition according to one of variants 1 to 7, characterized in that it is a concentrated solution comprising one or more electrolytes, in particular chosen from sodium, potassium, chlorine, magnesium or calcium, and bicarbonates.

Variant 9: Dialysis composition, according to one of variants 1 to 7, characterized in that it is a concentrated solution packaged in a bag suitable for a dialysis device, preferably a bag containing a solution volume of between 500 and 5000 mL and an amount of said polymer of between 5 and 5000 mg and preferably between 10 and 100 mg.

Variant 10: The invention relates to a method for capturing at least one toxic metal, such as lead, aluminum, chromium, nickel or cadmium, present in a dialysis fluid, comprising a step of adding to said dialysis fluid a sufficient amount of a polymer with a weight-average molecular mass of between 20 kDa and 1000 kDa bearing at least one agent for chelating at least one toxic metal such as lead, aluminum, chromium, nickel or cadmium, thus allowing capture of said toxic metal by chelation with the chelating agent of said polymer.

Variant 11: Capture method according to variant 10, characterized in that the toxic metal is aluminum.

Variant 12: Capture method according to variant 10, characterized in that the toxic metal is lead and the concentration in the dialysis fluid is less than 0.1 ppb.

Variant 13: Capture method according to variant 10, characterized in that the toxic metal is cadmium and the concentration in the dialysis fluid is less than 0.01 ppb.

Variant 14: Capture method according to variant 10 or 11, characterized in that at least 120 L of dialysis fluid are used in one patient dialysis.

Variant 15: Capture method according to one of variants 10 to 12, characterized in that the dialysis fluid is introduced into a tank of a dialysis device, which can be connected to an extracorporeal blood circulation device comprising a porous dialysis membrane.

Variant 16: Method according to variant 13, characterized in that the cut-off threshold of the porous membrane is below the polymer size.

Use of a polymer with a weight-average molecular mass of between 20 kDa and 1000 kDa bearing at least one agent for chelating at least one toxic metal such as lead, aluminum, chromium, nickel or cadmium to prepare a dialysis composition, for example for hemodialysis, hemodiafiltration or hemofiltration, in particular a composition according to one of variants 1 to 9.

### EXAMPLES

### Synthesis of the Polymers

### Preparation of MexCD1 (Chitosan@DOTAGA)

In step 1, 60 g of chitosan, 4 L of ultrapure water and 45 mL of glacial acetic acid are introduced into a 10 L reactor and stirred for 16 hours at a pH of 4.5±0.5. A pale yellow solution is obtained.

In step 2, 1.2 L of propane-1,2-diol are added to the pale yellow solution obtained in step 1, and stirring is maintained for 1 hr. A solution consisting of 14 mL acetic anhydride dissolved in 600 mL propane-1,2-diol is then added slowly over 10 min so as to obtain homogeneous acetylation along the polymer chain, and the reaction medium is kept stirring for 4 h.

The degree of acetylation may be determined by elemental analysis. The non-acetylated unit of the polysaccharide (unit B) has a molar mass of 161.2 g·mol⁻¹ (C₆NO₄H₁₁), while the acetylated unit (unit A) has a molar mass of 203.2 g·mol 1 (C₈NO₅H₁₃). The elemental analysis of the polysaccharide obtained on conclusion of acetylation step 2 is as follows: C 39.22%; H 7.55% and N 6.77%, corresponding to an acetylated unit (unit A) content of 40% (x=0.4).

In step 3, 2 L of the solution obtained in acetylation step 2 are placed in a reactor with stirring. 120 g of DOTAGA anhydride are then added and stirring is maintained for 16 h. On conclusion of this reaction, the solution is diluted 10-fold with ultrapure water and purified by tangential filtration using a 100 kDa membrane. After an initial step of re-concentration down to 16 L, the solution is filtered with 480 L of 0.1 M acetic acid solution at constant volume (16 L), followed by 320 L of ultrapure water and a further step of re-concentration down to 8 L. HPLC-UV is used to check that the DOTAGA has been removed. The peak at about 7 min corresponds to the polymer, while the peak at about 11 min corresponds to non-grafted DOTAGA. The solution, with a polysaccharide concentration of 10 g/L, was then filtered through a nylon filter (0.4 μm) before freeze-drying.

Proton NMR is used to determine the degree y of functionalization with DOTAGA on the polysaccharide, knowing the degree x of acetylation. The ungrafted, non-acetylated unit (unit B) consists of 7 protons, covalently bonded to carbon atoms, with a chemical shift of between 2.9 and 4.3 ppm. The acetylated unit (unit A) has these same 7 protons and also 3 protons, covalently bonded to a carbon atom, present on the acetyl, characterized by a chemical shift of between 2 and 2.2 ppm. Finally, the unit grafted with DOTAGA (unit C) includes 34 protons, covalently bonded to carbon atoms, of which 32 integrate between 2.9 and 4.3 ppm and 2 integrate between 2 and 2.2 ppm. By means of the integrations of the different multiplets, the NMR spectrum makes it possible to determine the values of y by means of the following equation:

$$[ Math . 1 ]$$

$$\frac{A r e {a}_{2.9 - 4.3}}{A r e {a}_{2 - 2.2}} = \frac{7 + 25 y}{3 x + 2 y}$$

The content of the grafted unit (unit C) is about 0.1.

Thus, the polysaccharide obtained has an A unit content of about 0.4 (x=0.4), a B unit content of about 0.5 (1−x−y=0.5) and a C unit content of about 0.1 (y=0.1).

The content of grafted units (unit C) may also be determined by fluorescence with europium. Specifically, europium exhibits luminescence mainly centered at about 590 (⁵D₀->⁷F₁) and 615 nm (⁵D₀->⁷F₂). This luminescence is extinguished when the europium ion is coordinated only with water molecules. The principle behind the method for determining the content of grafted units is to add increasing amounts of europium, so that it is chelated, and the luminescence increases. When all the chelation sites are filled, the luminescence reaches a plateau. In practice, the polysaccharide obtained on conclusion of step 3 was placed in an acetate buffer at pH 5, and a europium chloride salt dissolved in the acetate buffer was then added. An assay curve is then plotted by exciting at 396 nm and recording the emission at 590 nm. This assay allows a chelate amount of 0.4 μmol per mg of polymer to be determined, i.e. a content of about 10% (y=0.1).

### Synthesis of PEG@DOTAGA

The PEG used is an 8-Arm PEG Amine with a molecular weight of 40 kDa, purchased from Creative PEGWork. DOTAGA-anhydride (2,2′,2″-(10-(2,6-dioxotetrahydro-2H-pyran-3-yl)-1,4,7,10-tetraazacyclododecane-1,4,7-triyl)triacetic acid) was supplied by Chematech and used as received. The synthesis was performed in DMSO (Fischer Chemicals). 30 kDa VIVAFLOW cassettes for purification were purchased from Sartorius.

A 10 g mass of PEG was weighed out and placed in a 500 mL flask. A volume of 250 mL of DMSO was added and the solution was stirred and heated to 40° C. in a hot water bath. Once the polymer was fully dissolved, a 2.8 g mass of DOTAGA-anhydride was weighed out and placed in the PEG solution. The solution was stirred and heated at 40° C. for 1 h30 min. The solution was then purified with ultrapure water using a Vivaflow cassette with a 30 kDa cut-off threshold to a degree of purification of 1500. The purified product was then freeze-dried. The average mass of product recovered is 496 mg and 23.2 mg for 20 mL of solution.

HPLC-UV analysis of the PEG@DOTAGA at 295 nm was used to determine the purity of the product, based on the ratio of the polymer peak to the total peak area. This method makes it possible to conclude that the degree of purity of the PEG@DOTAGA is 97%.

The amount of grafted chelator is determined by UV-Visible spectrophotometric assay at 295 nm for DOTAGA. Increasing concentrations of copper (II) are added to a 1.0 g/L solution of PEG@DOTAGA in acetate buffer at pH 4.5 (0.1M ammonium acetate and 0.1M acetic acid). The absorbance measured at 295 nm is then plotted as a function of the copper concentration (in mmol/g). The observed break in slope indicates that the PEG@DOTAGA contains 0.14 mmol of DOTAGA per gram of polymer.

### Synthesis of PEG-EDTA and PEG-DTPA

The PEG used is an 8-Arm PEG Amine with a molecular weight of 40 kDa, purchased from Creative PEGWork. EDTA dianhydride (4-[2-(2,6-dioxomorpholin-4-yl)ethyl]morpholine-2,6-dione) and DTPA dianhydride (2-[bis [2-(2,6-dioxomorpholin-4-yl)ethyl]amino]acetic acid) were supplied by Chematech and used as received. The synthesis was performed in DMSO (Fischer Chemicals). 30 kDa VIVAFLOW cassettes for purification were purchased from Sartorius.

A 24 g/L EDTA dianhydride solution and a 35 g/L DTPA dianhydride solution were prepared in DMSO. For each synthesis, 10 mL of the chelator solution (24 g/L EDTA dianhydride or 35 g/L DTPA dianhydride) were placed in a 100 mL flask, and the solution was then stirred and heated to 40° C. in a hot water bath. 40 mL of a 6.25 g/L PEG solution in DMSO were placed in a dropping funnel. This solution was added dropwise. Once the addition was complete, the solution was stirred and heated at 40° C. for 1 h30 min. The solution was then purified with ultrapure water using a Vivaflow cassette with a 30 kDa cut-off threshold to a degree of purification of 20 000. The purified product was then freeze-dried. For PEG-EDTA and PEG-DTPA, the average mass of product recovered is 16.2 mg and 23.2 mg, for 5 mL of solution, respectively.

HPLC-UV analysis of PEG-EDTA and PEG-DTPA at 295 nm is used to determine the product purity by calculating the ratio of the polymer min peak to the total area of the peaks. This method makes it possible to conclude that the degrees of purity of PEG-EDTA and PEG-DTPA are 96% and 92%, respectively.

Comparison of the 1H NMR spectra of PEG and PEG-DTPA or PEG-EDTA after purification and freeze-drying confirms the grafting of the chelators.

The amount of grafted chelator is determined by UV-Visible spectrophotometric assay at 295 nm for EDTA and 275 nm for DTPA. Increasing concentrations of copper (II) are added to a 1.0 g/L solution of PEG-EDTA or PEG-DTPA in acetate buffer at pH 4.5 (0.1M ammonium acetate and 0.1M acetic acid). The absorbance measured at 295 nm (or 275 nm) is then plotted as a function of the copper concentration (in mmol/g). The observed breaks in slope indicate that PEG-EDTA and PEG-DTPA respectively contain 0.16 mmol EDTA and 0.12 mmol DTPA per gram polymer.

### Synthesis of PEG-DFO

The PEG used is an 8-Arm PEG Amine with a molecular weight of 40 kDa, purchased from Creative PEGWork. p-NCS-Bz-DFO (N1-hydroxy-N1-(5-(4-(hydroxy (5-(3-(4-isothiocyanatophenyl)thioureido)pentyl)amino)-4-oxobutanamido)pentyl)-N4-(5-(N-hydroxyacetamido) pentyl)succinamide) was supplied by Chematech and used as received. The synthesis was performed in DMSO (Fischer Chemicals). 30 kDa VIVAFLOW cassettes for purification were purchased from Sartorius.

A 1 g mass of PEG was weighed out and placed in a 250 mL flask. A volume of 100 mL of DMSO was added and the solution was stirred and heated to 40° C. in a hot water bath. Once the polymer was fully dissolved, a mass of 69.4 mg of p-NCS-Bz-DFO was weighed out and placed in the PEG solution. The solution was stirred and heated at 40° C. for 1 h30 min. The solution was then purified with ultrapure water using a Vivaflow cassette with a 30 kDa cut-off threshold to a degree of purification of 25 000. The purified product was then freeze-dried, the average mass of product recovered being 25 mg for 5 mL of solution.

1H NMR analysis of the PEG-DFO after purification and freeze-drying confirms the presence of grafted p-NCS-Bz-DFO. The characteristic peaks of benzene protons are found in the NMR spectrum of deferoxamine mesylate.

As in Example 1, the amount of grafted p-NCS-Bz-DFO can be determined by UV-Visible spectrophotometric assay at 430 nm. Increasing concentrations of iron (III) are added to a 1.0 g/L solution of PEG-DFO in acetate buffer at pH 4.5 (0.1M ammonium acetate and 0.1M acetic acid). The absorbance measured at 430 nm is then plotted as a function of the iron concentration, and a break in slope is observed at 7.9 μM iron. Since a DFO molecule can complex only one iron atom, 1 gram of polymer contains 7.9 μmol of DFO.

### Synthesis of PEG-DOTA

The PEG used is an 8-Arm PEG Amine with a molecular weight of 40 kDa, purchased from Creative PEGWork. DOTA-NHS (2-[4,10-bis(carboxymethyl)-7-[2-(2,5-dioxopyrrolidin-1-yl)oxy-2-oxoethyl]-1,4,7,10-tetraazacyclododec-1-yl]acetic acid) was supplied by Chematech and used as received. The synthesis was performed in DMSO (Fischer Chemicals). The 30 kDa PES membrane used for the purification was purchased from Sartorius and used with the SARTOFLOW SMART purification machine.

A 16.67 g/L PEG solution was prepared in DMSO. 30 mL of this solution were placed in a 100 mL flask, and the solution was stirred and heated to 40° C. in a hot water bath. A 7.5 g/L solution of DOTA-NHS was prepared in DMSO. 20 mL of the DOTA-NHS solution were placed in a dropping funnel. This solution was added dropwise. Once the addition was complete, the solution was stirred and heated at 40° C. for 1 h30 min. The solution was then purified in the SARTOFLOW SMART machine with a membrane having a 30 kDa cut-off threshold, using 8 L of ultrapure water, then 3 L of acetic acid and finally 2 L of water to return to neutral pH.

The amount of grafted chelator is determined by UV-Visible spectrophotometric assay at 275 nm. Increasing concentrations of copper (II) are added to a 1.0 g/L solution of PEG-DOTA in acetate buffer at pH 4.5 (0.1M ammonium acetate and 0.1M acetic acid). The absorbance measured at 275 nm is then plotted as a function of the copper concentration (in mmol/g). The observed break in slope indicates that PEG-DOTA contains 0.16 mmol of DOTA per gram of polymer.

After freeze-drying, PEG-DOTA was also characterized by 1H NMR, confirming the grafting of DOTA.

### Preparation of the Solutions

### Solution 1:10 g/L MexCD1

Freeze-dried MexCD1 was dissolved in ultrapure water to a concentration of 10 g/L.

### Solution 2:10 g/L PEG@DOTAGA

Freeze-dried PEG@DOTAGA was dissolved in ultrapure water to a concentration of 10 g/L.

### Solution 3:10 g/L PEG@EDTA

Freeze-dried PEG@EDTA was dissolved in ultrapure water to a concentration of 10 g/L.

### Solution 4:10 g/L PEG@DTPA

Freeze-dried PEG@DTPA was dissolved in ultrapure water to a concentration of 10 g/L.

### Solution 5: Concentrated Acetic Acid Standard for Hemodialysis

The composition of the concentrated acetic acid standard solution is as follows: 263 g/L NaCl, 3.35 g/L KCl, 6.24 g/L CaCl₂), 2.14 g/L MgCl₂, 10.8 g/L acetic acid and 45.0 g/L dextrose. This solution has a pH of 1.95.

### Solution 6: Concentrated Citric Acid Standard for Hemodialysis

The composition of the concentrated citric acid standard solution is as follows: 263 g/L NaCl, 3.35 g/L KCl, 6.24 g/L CaCl₂), 2.14 g/L MgCl₂, 1.11 g/L acetate, 6.92 g/L citric acid and 45.0 g/L dextrose. This solution has a pH of 1.08.

### Solution 7:0.1 M Acetate Buffer, pH 4.6

The acetate buffer solution contains 11.4 mL acetic acid (MS-grade), 15.4 g ammonium acetate and 2 L ultrapure water.

### Solution 8:0.010 M HEPES Buffer, pH 7.4

The buffer solution contains 2.383 g HEPES (Sigma-Aldrich) in 1000 ml of ultrapure water, pH adjusted with sodium hydroxide.

### Experimental Protocols:

### Protocol 1: High Pressure Liquid Chromatography (HPLC)

The eluent used is acetate buffer (solution 7), 10 μL of sample are injected and eluted at a flow rate of 0.8 mL/min. The column used is an SEC Polysep GFC-P 4000 column, and detection is performed with a UV detector at wavelengths of 250 nm and 295 nm.

### Protocol 2: High Pressure Liquid Chromatography-Mass Spectroscopy (HPLC-MS)

The eluent used is acetate buffer (solution 7 prepared with MS-Grade certified products), the injection volume is 10 μL and the flow rate is set at 0.4 mL/min. The column used is an SEC Polysep GFC-P 4000 column, and the HPLC is coupled to a mass spectrometer (ICP-MS) for detection of the isotopes of interest.

### Protocol 3: Ultrafiltration Experiment Using Vivaspin Tubes

Various Vivaspin centrifugation tubes were used: Vivaspin 20 mL with a 30 kDa cutoff threshold membrane or Vivaspin 6 mL with a 30 kDa or 10 kDa cutoff threshold membrane. All the equipment was precleaned with a 21 mM EDTA solution and then twice with ultrapure water (18.2 MΩ) to remove any trace of metallic contamination. The centrifugation was performed at a speed of 4000 rpm for 10 min. After each centrifugation cycle, the liquid that had passed through the membrane was collected for each sample, this part is called the subnatant, and the centrifugation tubes were refilled with the initial solution. This was repeated until all the initial solution was used. Ideally, at least 1 mL of supernatant remained above the membrane and was analyzed by ICP-MS to determine the exact concentration factor of the experiment.

Samples of the initial solution before centrifugation, the subnatant and the supernatant at the end of the experiment were analyzed by ICP-MS. The samples were diluted at least 10-fold with 1% nitric acid solution. An internal standard (indium) was added to each sample to achieve a final indium concentration of 2 ppb. The analyses were performed in KED (Kinetic Energy Discrimination) mode with a stream of helium in the chamber. The isotopes detected were as follows: ²⁰⁸Pb, ²⁰⁶Pb, ¹¹¹Cd, ¹¹²Cd, ¹¹⁴Cd, ⁶³Cu, ⁶⁵Cu, ²⁷Al.

A calibration curve between 0.02 and 10 ppb cadmium and 0.1 and 50 ppb lead was produced. This calibration allows reliable conversion from the number of counts per second to a concentration (ppb) for each sample, using the software.

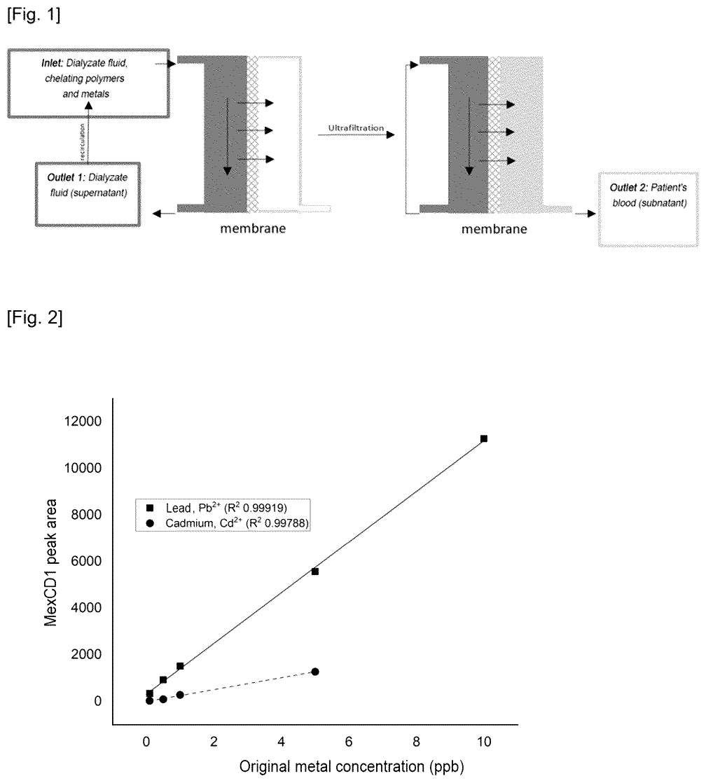

The principle of Vivaspin ultrafiltration is shown in FIG. 1.

### Example 1: Chelation of Pb²⁺ and Cd²⁺ with MexCD1 and PEG@DOTAGA

The samples were created using solution 8. Starting with a 10 ppm metal mixture of Pb²⁺ and Cd²⁺ in 5% HNO₃ purchased from SCP Science, and also polymer solutions 1 and 2, six samples per polymer were created and analyzed by HPLC-MS according to protocol 2. The composition of the samples is given in table 1.

TABLE 1

| | | Metal concentration |
|---|---|---|
| Sample | Polymer | (ppb) |
| 1 | 0.1 g/L MexCD1 | 0 |
| 2 | | 0.1 |
| 3 | | 0.5 |
| 4 | | 1 |
| 5 | | 5 |
| 6 | | 10 |
| 7 | 0.1 g/L | 0 |
| 8 | PEG@DOTAGA | 0.1 |
| 9 | | 0.5 |
| 10 | | 1 |
| 11 | | 5 |
| 12 | | 10 |

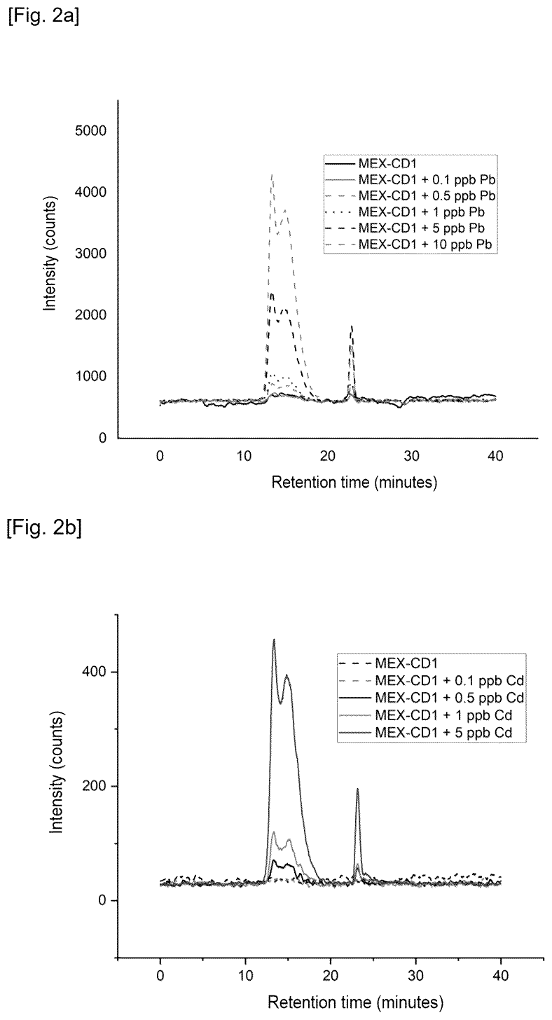

FIG. 2 shows that lead and cadmium are captured by the functionalized polymer product, MEXCD1, in the ppb concentration range.

Specifically, in FIG. 2, the y-axis shows the polymer peak area and the x-axis the original metal concentration. Linear regression shows that the capture of lead and cadmium increases in a linear manner with the amount of polymer introduced (R2 greater than 0.99).

FIGS. 2a and 2b below show HPLC/SEC chromatograms coupled with ICP-MS for the detection of lead or cadmium. The peaks correspond to polymer peaks with retention times of about 15 minutes. The increase in the area of the peak corresponding to the polymer with increasing initial lead or cadmium concentration illustrates the increasingly strong capture of lead and cadmium as a function of the concentrations introduced.

### Example 2: Chelation of Lead Pb²⁺ by MEX-CD1 in Acetic and Citric Acid Concentrates

77 ppb of Pb²⁺ were added to solution 5 and 26 ppb of Pb²⁺ were added to solution 6. These solutions, in addition to solutions 1 (MEX-CD1 at 10 g/L) and 7 (MS Grade acetate buffer) and ultrapure water, were used to create different acidic environments with 0.1 g/L of MEX-CD1. Complexation was tested under three conditions: in concentrated acid (pH 2 for acetic acid and pH 1.2 for citric acid), at pH 5 and at pH 7.4. The pH was adjusted with sodium hydroxide and the samples were analyzed by HPLC-MS according to protocol 2. The composition of the samples is given in Tables 2 and 3.

TABLE 2

| | Acid dilution | |
|---|---|---|
| Sample | factor | pH |
| Concentrated acetic acid | 1 | 2 |
| Concentrated acetic acid + | 1 | 2 |
| 0.1 g/L MexCD1 | | |
| Acetic acid + 0.1 g/L MexCD1 | 5 | 5 |
| Acetic acid + 0.1 g/L MexCD1 | 5 | 7.4 |

TABLE 3

| | Acid dilution | |
|---|---|---|
| Sample | factor | pH |
| Concentrated citric acid | 1 | 1.2 |
| Concentrated citric acid + | 1 | 1.2 |
| 0.1 g/L MexCD1 | | |
| Citric acid + 0.1 g/L MexCD1 | 5 | 5 |
| Citric acid + 0.1 g/L MexCD1 | 5 | 7.4 |

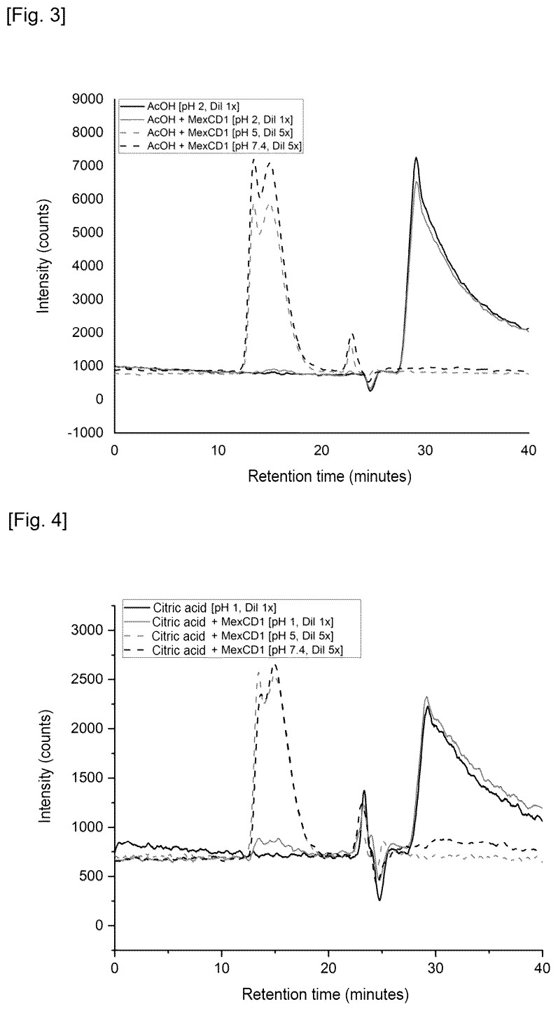

FIGS. 3 and 4 show that the polymer dispersed at 0.1 g/L directly in a solution representative of an acid concentrate (acetic acid or citric acid) is efficient for capturing lead as soon as the dialysis fluid begins to formulate. At pH 5 and 7.4, a large proportion of the lead is thus already captured and associated with the polymer, as shown by the HPLC/SEC-ICP-MS chromatogram (Pb detection) with the increase in the peak at the 15 min retention time corresponding to the polymer.

### Example 3: Chelation of Lead Pb²⁺ by PEG@DOTAGA in Acetic and Citric Acid Concentrates

77 ppb of Pb²⁺ were added to solution 5 and 26 ppb of Pb²⁺ were added to solution 6. These solutions, in addition to solutions 2 (10 g/L PEG@DOTAGA) and 7 (MS Grade acetate buffer) and ultrapure water, were used to create different acidic environments with 0.1 g/L PEG@DOTAGA. Complexation was tested under three conditions: in concentrated acid (pH 2 for acetic acid and pH 1.2 for citric acid), at pH 5 and at pH 7.4. The pH was adjusted with sodium hydroxide and the samples were analyzed by HPLC-MS according to protocol 2. The composition of the samples is given in Tables 4 and 5.

TABLE 4

| | Acid dilution | |
|---|---|---|
| Sample | factor | pH |
| Concentrated acetic acid | 1 | 2 |
| Concentrated acetic acid + 0.1 g/L | 1 | 2 |
| PEG@DOTAGA | | |
| Acetic acid + 0.1 g/L PEG@DOTAGA | 5 | 5 |
| Acetic acid + 0.1 g/L PEG@DOTAGA | 5 | 7.4 |

TABLE 5

| | Acid dilution | |
|---|---|---|
| Sample | factor | pH |
| Concentrated citric acid | 1 | 1.2 |
| Concentrated citric acid + | 1 | 1.2 |
| 0.1 g/L PEG@DOTAGA | | |
| Citric acid + 0.1 g/L PEG@DOTAGA | 5 | 5 |
| Citric acid + 0.1 g/L PEG@DOTAGA | 5 | 7.4 |

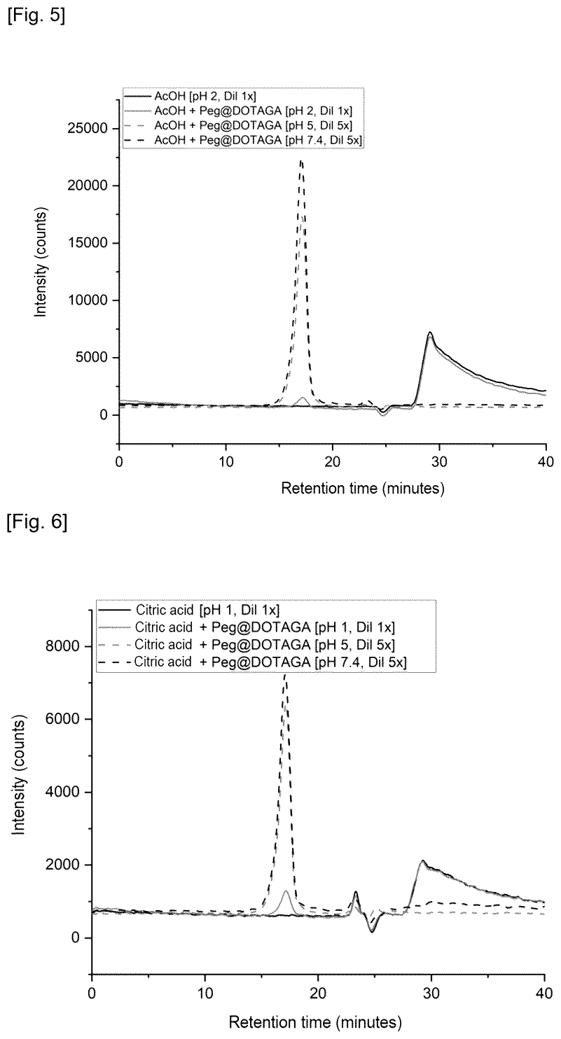

FIGS. 5 and 6 show that the polymer dispersed at 0.1 g/L directly in a solution representative of an acid concentrate (acetic acid or citric acid) is efficient for capturing lead as soon as the dialysis fluid begins to formulate. At pH 5 and 7.4, a large proportion of the lead is thus already captured and associated with the polymer, as shown by the HPLC/SEC-ICP-MS chromatogram (Pb detection) with the increase in the peak at the 17 min retention time corresponding to the polymer.

### Example 4: Chelation of Lead Pb²⁺ by MEX-CD1 Via Ultrafiltration

The samples were prepared from ultrapure water, solution 1 and a 50 000 ppm Pb²⁺ stock solution in 5% HNO₃ nitric acid to obtain a sample with a total volume of 100 mL. Following protocol 3, 90 mL of each solution were centrifuged through 20 mL Vivaspins with a cutoff threshold of 30 kDa. The volume of the final supernatant was about 10 mL, resulting in concentration of the initial sample by a factor of 9. The composition of the samples and the results are given in Table 6.

TABLE 6

| | | | | Results | | |
|---|---|---|---|---|---|---|
| Preparation of the sample | | | | Original | Supernatant | Subnatant |
| | Concentration | Concentration | | [Pb²⁺] | [Pb²⁺] | [Pb²⁺] |
| Sample | MexCD1 | Pb²⁺ (ppb) | pH | (ppb) | (ppb) | (ppb) |
| 1 | 0 | 10 | 6.40 | 5.418 | | 0.855 |
| 2 | 10 mg/L | 0 | 5.79 | 0.024 | 0.048 | 0.013 |
| 3 | | 0.1 | 5.73 | 0.113 | 0.543 | 0.019 |
| 4 | | 5 | 6.03 | 4.412 | 22.65 | 0.103 |
| 5 | | 10 | 5.78 | 8.841 | 50.25 | 0.102 |

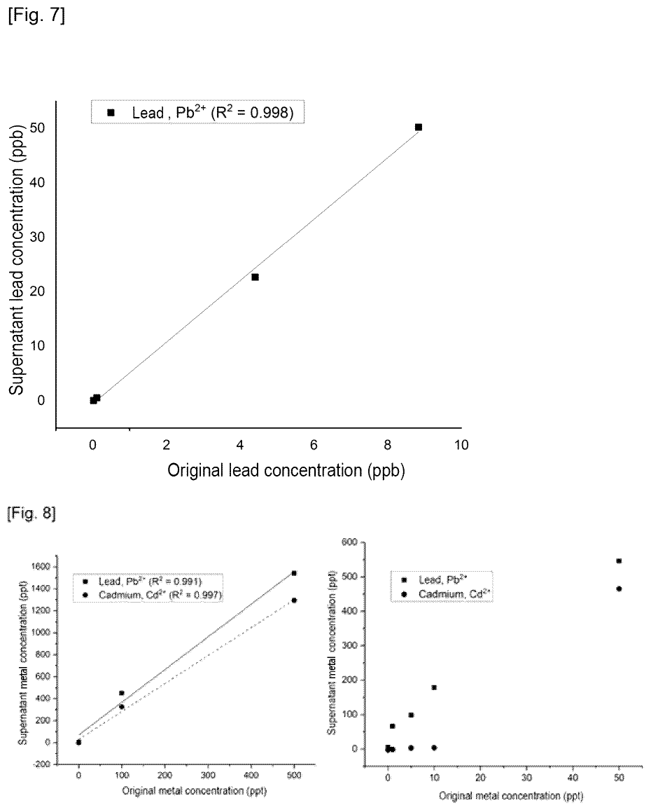

The HPLC/SEC-ICP-MS chromatogram (Pb detection) shown in FIG. 7 corresponds to an initial lead concentration of about 5 ppb, purified by tangential filtration through a 30 kDa membrane. If 10 mg/L of MEX-CD1 are dissolved, 8 times less lead passes through the membrane compared with the same experiment performed without MEX-CD1. The product shows efficacy at very low concentrations (0.1 ppb).

### Example 5: Chelation of Metals in Trace Amount by MEX-CD1 Via Ultrafiltration

Two metal solutions purchased from SCP Science, one of iron at 1000 ppm in 5% HNO₃ and a mixture of various metals (Al²⁺, Mn²⁺, Pb²⁺, Cd²⁺, Cu²⁺, Zn²⁺) at 10 ppm in 5% HNO₃, were used in addition to solution 1 and ultrapure water to create samples with a final volume of 800 mL. According to protocol 3, about 350 mL of each sample were centrifuged through 20 mL Vivaspins having a membrane with a cutoff threshold of 30 kDa. The volume of the final supernatant is about 10 mL, i.e. reconcentration of the initial sample by a factor of 35. The composition of the samples is given in table 7.

TABLE 7

| Sample | Concentration MexCD1 | Concentration of metals (ppt) | pH |
|---|---|---|---|
| 1 | 0 | 100 | ~6.5 |
| 2 | 1 mg/L | 0 | |
| 3 | | 1 | |
| 4 | | 5 | |
| 5 | | 10 | |
| 6 | | 50 | |
| 7 | | 100 | |
| 8 | | 500 | |
| 9 | | 1000 | |

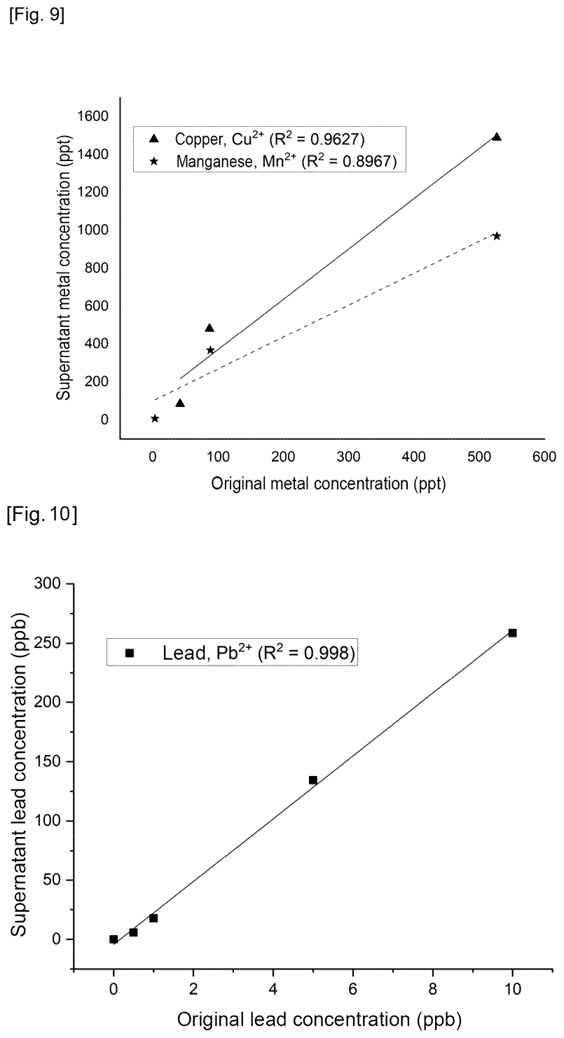

FIGS. 8 and 9 show capture efficiencies as low as 0.05 ppb (50 ppt) for lead and cadmium by ICP/MS measurement. Manganese and copper are also captured. With lead, even in the 0.01 ppb range, capture efficacy is observed.

### Example 6: Chelation of Pb²⁺ by PEG@DOTAGA via Ultrafiltration

The samples were prepared from solution 2, ultrapure water and a 50 000 ppm Pb²⁺ solution in 5% HNO₃ purchased from SCP Science, with a final sample volume of 100 mL. According to protocol 3, 99 mL of each sample were centrifuged through 6 mL Vivaspins with a pore size of 10 kDa. The final supernatant has a volume of 1 mL, i.e. reconcentration of the initial sample by a factor of 99. The composition of the samples and the results are given in Table 8.

TABLE 8

| | | | | Results | | |
|---|---|---|---|---|---|---|
| Preparation of the sample | | | | Original | Supernatant | Subnatant |
| | Concentration | Concentration Pb²⁺ | | [Pb²⁺] | [Pb²⁺] | [Pb²⁺] |
| Sample | PEG@DOTAGA | (ppb) | pH | (ppb) | (ppb) | (ppb) |
| 1 | 0 | 5 | 4.62 | 4.343 | | 2.888 |
| 2 | 10 mg/L | 0 | 6.74 | 0.005 | 0.025 | 0.007 |
| 3 | | 0.5 | 4.67 | 0.492 | 5.705 | 0.259 |
| 4 | | 1 | 4.16 | 0.989 | 17.68 | 0.352 |
| 5 | | 5 | 4.62 | 4.904 | 134.34 | 0.945 |
| 6 | | 10 | 4.12 | 9.607 | 258.48 | 1.827 |

FIG. 10 shows the efficacy of PEG@DOTAGA in capturing lead in the ppb range. For an initial lead concentration of between 0.5 and 10 ppb, after the ultrafiltration experiments, a 10- to 20-fold higher concentration of lead was measured in the supernatant solution by ICP/MS. The solution was thus over-concentrated in lead, and part of the lead was sequestered to prevent it from passing through the porous membrane.

### Example 7: Chelation of Metals in Trace Amounts by MEX-CD1 in Dialysis by Ultrafiltration

Solution 7 was diluted to 10 mM and 100 mg/L of Ca²⁺ (from CaCl₂), Sigma-Aldrich) were added to this solution to reproduce a dialysis-like environment. The samples were then prepared from this solution, solution 1 and a mixture of metals (Al²⁺, Mn²⁺, Pb²⁺, Cd²⁺, Cu²⁺ and Zn²⁺) at 10 ppm in 5% HNO₃ purchased from SCP Science, so as to obtain a final sample volume of 31 mL. According to protocol 3, 30 mL of each solution were centrifuged through Vivaspin 6 mL membranes with a 30 kDa cutoff threshold. The final supernatant has a volume of 1 mL, i.e. reconcentration of the initial sample by a factor of 30.

This situation presents conditions close to dialysis conditions in terms of ionic concentrations, which is particularly important since calcium Ca²⁺ has strong interactions with the chelator DOTAGA. The concentrations of Ca²⁺ in blood and dialyzate are close to 100 mg/L.

The composition of the samples and the results are given in Tables 9 and 10.

TABLE 9

| | | | Desired metal | |
|---|---|---|---|---|
| | Concentration | Concentration | concentration | |
| Sample | Ca²⁺ | MexCD1 | (ppb) | pH |
| 1 | 100 mg/L | 0 | 1 | 4.65 |
| 2 | | 10 mg/L | 0 | 4.68 |
| 3 | | | 0.01 | 4.68 |
| 4 | | | 0.1 | 4.61 |
| 5 | | | 1 | 4.60 |

TABLE 10

| | Pb²⁺ | | | Cd²⁺ | | | Cu²⁺ | | |
|---|---|---|---|---|---|---|---|---|---|
| | (ppb) | | | (ppb) | | | (ppb) | | |
| Sample | Original | Supernatant | Subnatant | Original | Supernatant | Subnatant | Original | Supernatant | Subnatant |
| 1 | 1.111 | 1.217 | 1.108 | 1.022 | 1.163 | 1.056 | 1.380 | 1.490 | 1.333 |
| 2 | 0.157 | 0.577 | 0.107 | 0.083 | 0.098 | 0.096 | 0.515 | 1.659 | 0.264 |
| 3 | 0.270 | 0.663 | 0.188 | 0.081 | 0.212 | 0.080 | 0.534 | 3.096 | 0.244 |
| 4 | 0.595 | 1.374 | 0.072 | 0.162 | 0.412 | 0.158 | 0.546 | 1.566 | 0.305 |
| 5 | 1.168 | 2.697 | 1.055 | 1.096 | 2.708 | 1.018 | 1.503 | 4.073 | 1.203 |

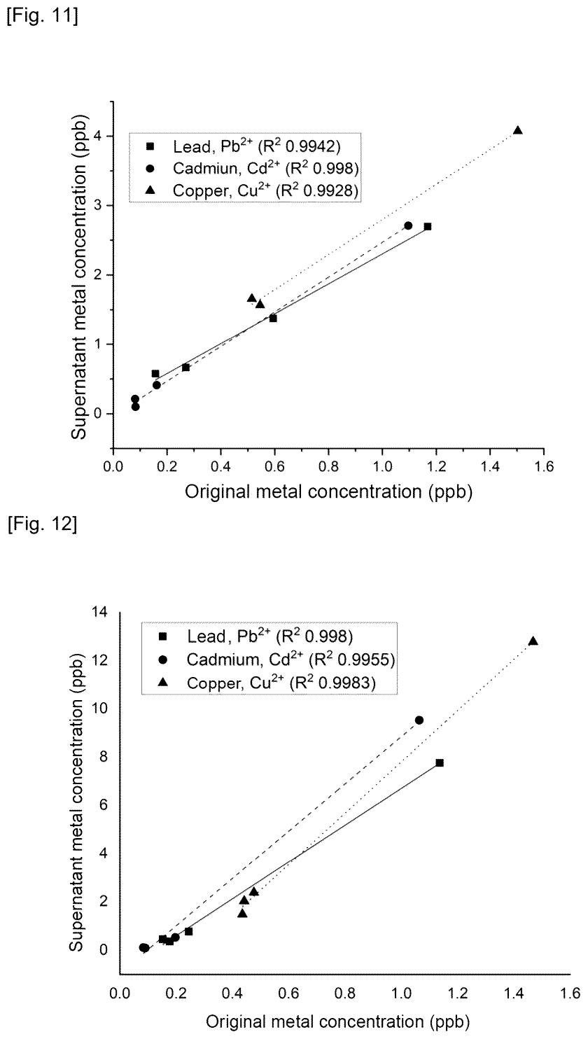

FIG. 11 shows the capture of lead as a function of the original lead concentration. When MEX-CD1 polymers are placed at a concentration of 100 mg/mL, heavy metal retention is possible even in the presence of 100 ppm of calcium and when they are placed in trace amounts at a concentration below the ppb level.

### Example 8: Chelation of Metals in Trace Amounts by PEG@DOTAGA Under Ultrafiltration Dialysis Conditions

Solution 7 was diluted to 10 mM and 100 mg/L of Ca²⁺ (from CaCl₂), Sigma-Aldrich) were added to this solution to reproduce a dialysis-like environment. The samples were then prepared from this solution, solution 1 and a mixture of metals (Al²⁺, Mn²⁺, Pb²⁺, Cd²⁺, Cu²⁺ and Zn²⁺) at 10 ppm in 5% HNO₃ purchased from SCP Science, so as to obtain a sample with a final volume of 31 mL. According to protocol 3, 30 mL of each solution were centrifuged through the 30 kDa membranes of 6 mL Vivaspins. The final supernatant has a volume of 1 mL, i.e. reconcentration of the initial sample by a factor of 30.

This situation presents conditions close to dialysis conditions in terms of ionic concentrations, which is particularly important since calcium Ca²⁺ has strong interactions with the chelator DOTAGA. The concentrations of Ca²⁺ in blood and dialyzate are close to 100 mg/L.

The composition of the samples and the results are given in Tables 11 and 12.

TABLE 11

| | | | Desired metal | |
|---|---|---|---|---|
| | Concentration | Concentration | concentration | |
| Sample | Ca²⁺ | PEG@DOTAGA | (ppb) | pH |
| 1 | 100 mg/L | 0 | 1 | 4.65 |
| 2 | | 10 mg/L | 0 | 4.71 |
| 3 | | | 0.01 | 4.71 |
| 4 | | | 0.1 | 4.64 |
| 5 | | | 1 | 4.64 |

TABLE 12

| | Pb²⁺ | | | Cd²⁺ | | | Cu²⁺ | | |
|---|---|---|---|---|---|---|---|---|---|
| | (ppb) | | | (ppb) | | | (ppb) | | |
| Sample | Original | Supernatant | Subnatant | Original | Supernatant | Subnatant | Original | Supernatant | Subnatant |
| 1 | 1.111 | 1.217 | 1.108 | 1.022 | 1.163 | 1.056 | 1.380 | 1.490 | 1.333 |
| 2 | 0.153 | 0.454 | 0.073 | 0.081 | 0.107 | 0.084 | 0.476 | 2.381 | 0.208 |
| 3 | 0.177 | 0.359 | 0.193 | 0.093 | 0.115 | 0.089 | 0.436 | 1.484 | 0.227 |
| 4 | 0.245 | 0.771 | 0.761 | 0.188 | 0.503 | 0.163 | 0.442 | 2.029 | 0.311 |
| 5 | 1.137 | 7.761 | 0.121 | 1.040 | 9.402 | 0.624 | 1.469 | 12.781 | 0.664 |

FIG. 12 shows that in the presence of 100 ppm of calcium, the PEG@DOTAGA product added at 100 mg/L is capable of sequestering lead and cadmium, even at low initial contents of between 0.1 and 1 ppb. Thus, relative to the reference (without the product) at an initial lead or cadmium concentration of about 1 ppb, almost 10 times less lead (9.15 times less lead in the subnatant) and almost half as much cadmium (1.69 times less cadmium in the subnatant) can pass through the membrane.

### Example 9: Chelation of Metals in Trace Amounts by PEG@EDTA Under Ultrafiltration Dialysis Conditions

Solution 7 was diluted to 10 mM and 100 mg/L of Ca²⁺ (from CaCl₂), Sigma-Aldrich) were added to this solution to reproduce a dialysis-like environment. The samples were then prepared from this solution, solution 1 and a mixture of metals (Al²⁺, Mn²⁺, Pb²⁺, Cd²⁺, Cu²⁺ and Zn²⁺) at 10 ppm in 5% HNO₃ purchased from SCP Science, so as to obtain a sample with a final volume of 31 mL. According to protocol 3, 30 mL of each solution were centrifuged through the 30 kDa membranes of 6 mL Vivaspins. The final supernatant has a volume of 1 mL, i.e. reconcentration of the initial sample by a factor of 30.

The composition of the samples and the results are given in Tables 13 and 14.

TABLE 13

| | | | Desired metal | |
|---|---|---|---|---|
| | Concentration | Concentration | concentration | |
| Sample | Ca²⁺ | PEG@EDTA | (ppb) | pH |
| 1 | 100 mg/L | 0 | 1 | 4.65 |
| 2 | | 10 mg/L | 0 | 4.68 |
| 3 | | | 0.01 | 4.69 |
| 4 | | | 0.1 | 4.62 |
| 5 | | | 1 | 4.61 |

TABLE 14

| | Pb²⁺ | | | Cd²⁺ | | | Cu²⁺ | | |
|---|---|---|---|---|---|---|---|---|---|
| | (ppb) | | | (ppb) | | | (ppb) | | |
| Sample | Original | Supernatant | Subnatant | Original | Supernatant | Subnatant | Original | Supernatant | Subnatant |
| 1 | 1.111 | 1.217 | 1.108 | 1.022 | 1.163 | 1.056 | 1.380 | 1.490 | 1.333 |
| 2 | 0.183 | 1.482 | 0.101 | 0.090 | 0.194 | 0.089 | 1.627 | 4.493 | 0.279 |
| 3 | 0.194 | 1.853 | 0.073 | 0.092 | 0.407 | 0.088 | 0.544 | 5.683 | 0.145 |
| 4 | 0.270 | 2.052 | 0.112 | 0.198 | 1.237 | 0.128 | 0.590 | 4.709 | 0.227 |
| 5 | 1.228 | 13.507 | 0.463 | 1.182 | 13.715 | 0.389 | 1.578 | 18.057 | 0.511 |

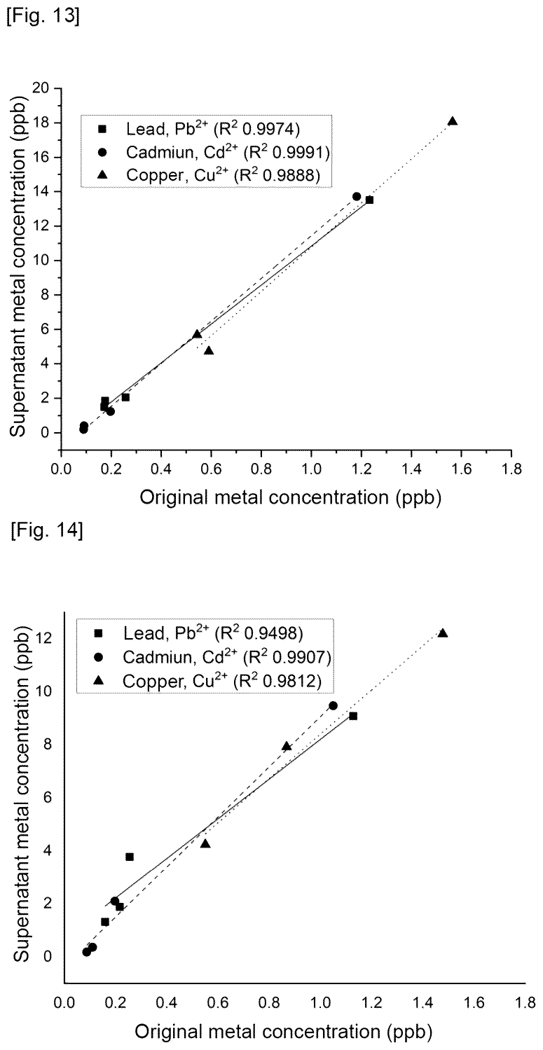

FIG. 13 shows that, in the presence of calcium, the product PEG@EDTA also captures part of the lead and cadmium, preventing it from passing the diafiltration membrane and over-concentrating the supernatant solution, between 0.1 and 1 ppb. Thus at 0.1 ppb of cadmium or 0.2 ppb of lead and in the presence of 100 ppm of calcium, after one diafiltration cycle, the supernatant was concentrated by 4.4 in cadmium and by 9.5 in lead.

### Example 10: Chelation of Metals in Trace Amounts by PEG@DTPA Under Ultrafiltration Dialysis Conditions

Solution 7 was diluted to 10 mM and 100 mg/L of Ca²⁺ (from CaCl₂), Sigma-Aldrich) were added to this solution to reproduce a dialysis-like environment. The samples were then prepared from this solution, solution 1 and a mixture of metals (Al²⁺, Mn²⁺, Pb²⁺, Cd²⁺, Cu²⁺ and Zn²⁺) at 10 ppm in 5% HNO₃ purchased from SCP Science, so as to obtain a sample with a final volume of 31 mL. According to protocol 3, 30 mL of each solution were centrifuged through the 30 kDa membranes of 6 mL Vivaspins. The final supernatant has a volume of 1 mL, i.e. reconcentration of the initial sample by a factor of 30.

The composition of the samples and the results are given in Tables 15 and 16.

TABLE 15

| | | | Desired metal | |
|---|---|---|---|---|
| | Concentration | Concentration | concentration | |
| Sample | Ca²⁺ | PEG@DTPA | (ppb) | pH |
| 1 | 100 mg/L | 0 | 1 | 4.65 |
| 2 | | 10 mg/L | 0 | 4.69 |
| 3 | | | 0.01 | 4.69 |
| 4 | | | 0.1 | 4.62 |
| 5 | | | 1 | 4.62 |

TABLE 16

| | Pb²⁺ | | | Cd²⁺ | | | Cu²⁺ | | |
|---|---|---|---|---|---|---|---|---|---|
| | (ppb) | | | (ppb) | | | (ppb) | | |
| Sample | Original | Supernatant | Subnatant | Original | Supernatant | Subnatant | Original | Supernatant | Subnatant |
| 1 | 1.111 | 1.217 | 1.108 | 1.022 | 1.163 | 1.056 | 1.380 | 1.490 | 1.333 |
| 2 | 0.174 | 1.316 | 0.076 | 0.088 | 0.177 | 0.077 | 0.551 | 4.241 | 0.134 |
| 3 | 0.226 | 1.879 | 0.065 | 0.110 | 0.362 | 0.083 | 2.007 | 5.723 | 0.133 |
| 4 | 0.277 | 3.770 | 0.076 | 0.198 | 2.095 | 0.093 | 0.868 | 7.911 | 0.250 |
| 5 | 1.147 | 9.078 | 0.769 | 1.051 | 9.467 | 0.746 | 1.478 | 12.179 | 0.936 |

FIG. 14 shows that the product PEG@DTPA also offers advantageous efficacy for sequestering lead and cadmium, even in the presence of 100 ppm of calcium.

### Example 11: Stability of the Polymers in Acidic Medium

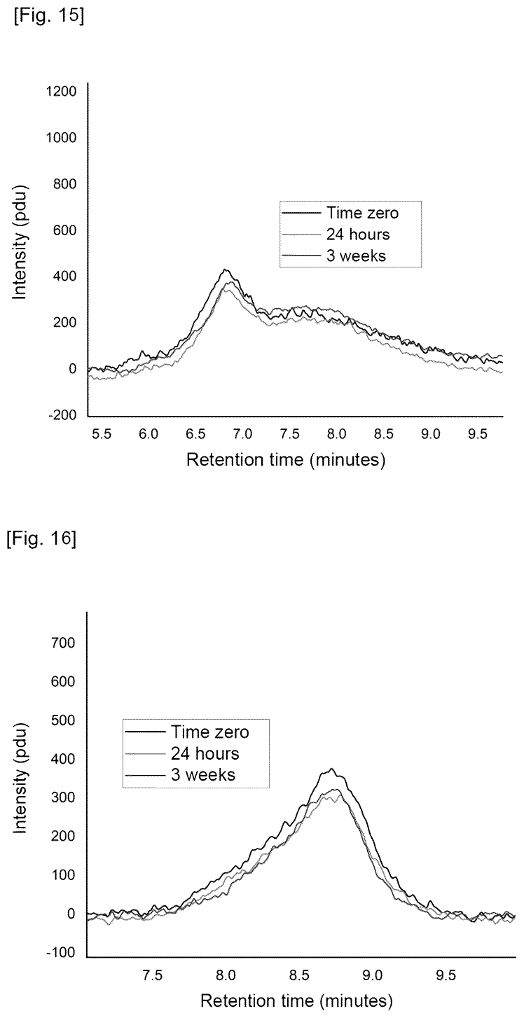

FIGS. 15 and 16 show that the product can be dispersed directly in an acid concentrate to be used. Thus, 1 g/L of MEX-CD1 and 1 g/L of PEG@DOTAGA were placed in a solution similar to an acid concentrate of the type such as citric acid and salts thereof. After several weeks of storage, no modification of the product was observed, the product did not appear to have degraded and retained its capture capacity (tested here with copper) after 3 weeks.

### Example 12: Complexation of Cd²⁺ and Pb²⁺ by PEG-DOTA in Acidic Medium

PEG-DOTA was dissolved in ultrapure water to a concentration of 1 g/L. Standard solutions of lead and cadmium at 1000 ppm in 5% nitric acid (purchased from SCP Science) were diluted 2000-fold in 0.01M hydrochloric acid.

PEG-DOTA was diluted to 10 mg/L in dilute acetate buffer (0.01 M ammonium acetate and 0.01 M acetic acid) and 1 ppb of each of the metals (Pb and Cd) was added to the sample. The pH was adjusted with 0.1M or 1M hydrochloric acid to pH 4, 3, 2 or 1.

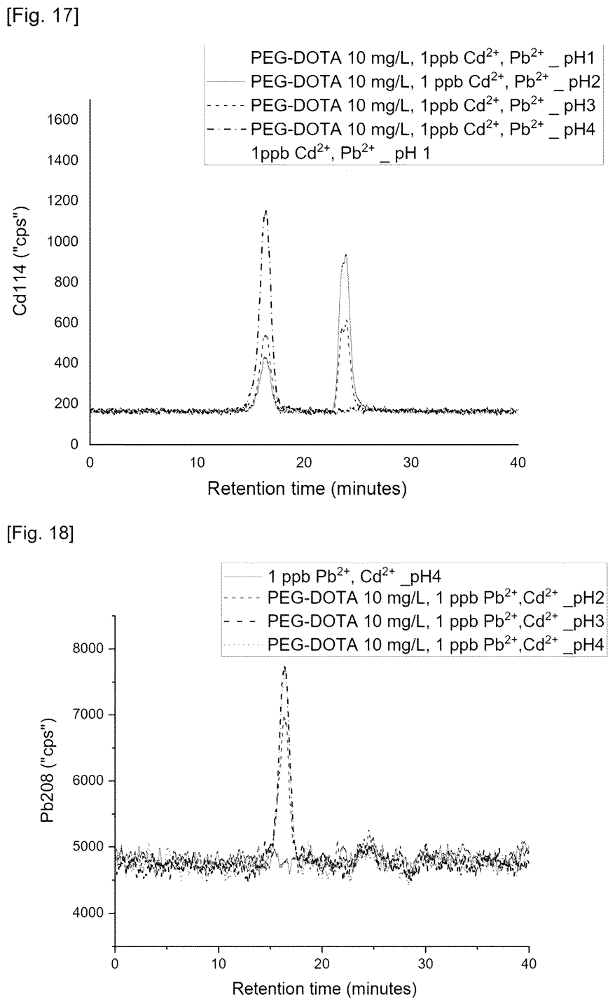

HPLC-MS analysis of the samples shows that PEG-DOTA at 10 mg/L is capable of complexing cadmium at all four pH levels tested. Considering that the total area of the peaks of free and polymer-bound cadmium corresponds to the total amount of metal added, PEG-DOTA is capable of complexing over 26% of the cadmium at pH 1 and 2. This percentage rises to 50% and 100% at pH 3 and 4, respectively (FIG. 17). As regards lead, no peak is visible at pH 1 (not shown here), whereas a polymer-bound lead peak is observed at 17 min at pH 2, 3 and 4, confirming the capture of lead by PEG-DOTA (FIG. 18).

### Example 13: Complexation of Aluminum by PEG-DFO in the Presence of Citrate

Freeze-dried PEG-DFO was dissolved in ultrapure water to a concentration of 10 g/L. The standard solution of 1000 ppm of aluminum in 5% nitric acid (purchased from SCP Science) was diluted 200-fold in 0.01 M hydrochloric acid. A 2.0 mM citric acid solution was prepared in ultrapure water. The samples were prepared in acetate buffer (0.1 M ammonium acetate and 0.1 M acetic acid, pH 4.6).

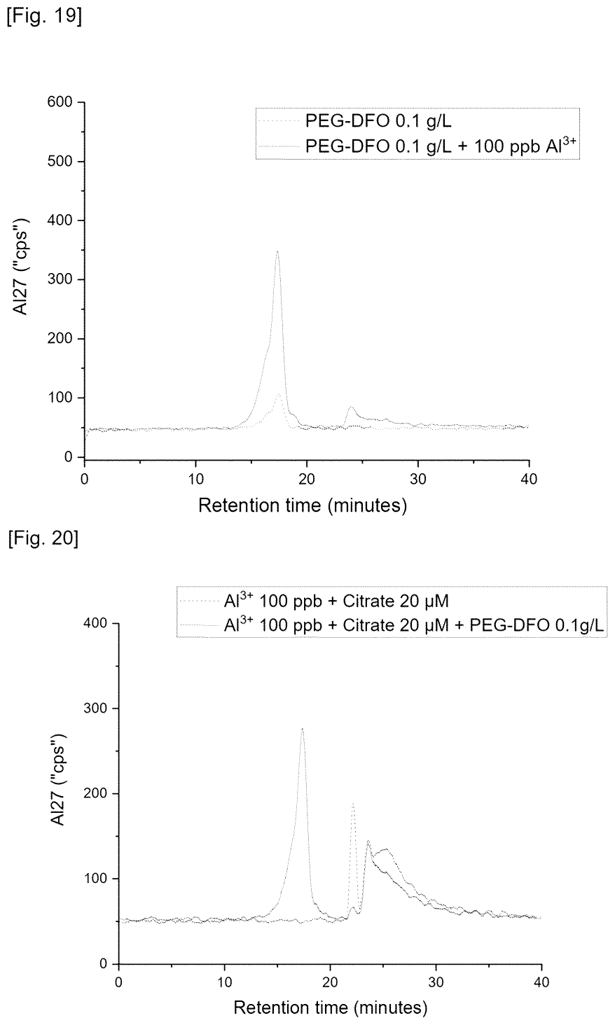

PEG-DFO was diluted to 0.1 g/L in acetate buffer and 100 ppb of aluminum were added to the sample. Where samples contained citrate, this was added first, followed by aluminum and then polymer, so as to study the ability of PEG-DFO to extract aluminum already complexed by citrate. The samples were analyzed by HPLC-MS to observe the distribution of aluminum on the different species present (PEG-DFO, citrate or free aluminum). HPLC-MS analysis showed a polymer-bound aluminum peak at a retention time of 17 min and a very small free aluminum peak at 24 min, confirming the complexation of aluminum by the polymer (FIG. 19). Considering that the total area of the peaks of free and polymer-bound aluminum (from which the area of the polymer without added aluminum was subtracted) corresponds to 100 ppb of added metal, PEG-DFO is capable of complexing over 85% of the aluminum. In the presence of 20 M citrate and 0.1 g/L PEG-DFO, aluminum is transferred from citrate (peak at 22 min) to the polymer (peak at 17 min) (FIG. 20). As previously, if we consider the actual total area of the peaks (polymer, citrate and free metal) to be 100 ppb of aluminum, PEG-DFO is capable of capturing 45% of the total aluminum and over 90% of the aluminum complexed by citrate.

### Example 14: Extraction of EDTA-Complexed Aluminum by PEG-DFO

Freeze-dried PEG-DFO was dissolved in ultrapure water to a concentration of 10 g/L. The standard solution of 1000 ppm of aluminum in 5% nitric acid (purchased from SCP Science) was diluted 200-fold in 0.01M hydrochloric acid. An 8 mM EDTA solution was prepared in ultrapure water. The samples were prepared in a buffered solution of Hemosol B0 diluted 100-fold. Reference samples of aluminum-free polymer and aluminum complexed with 1.1 molar equivalents of EDTA, i.e. 1.189 ppm EDTA, without polymer were prepared. The composition of the various samples is detailed in Table 17.

TABLE 17

| Sample | [PEG-DFO] (mg/L) | [Al³⁺] (ppb) | [EDTA] (ppm) | pH |
|---|---|---|---|---|
| 1 | 0 | 100 | 1.189 | 6.23 |
| 2 | 100 | 100 | 2.162 | 6.01 |
| 3 | 100 | 100 | 1.189 | 6.11 |
| 4 | 100 | 0 | 0 | 7.96 |

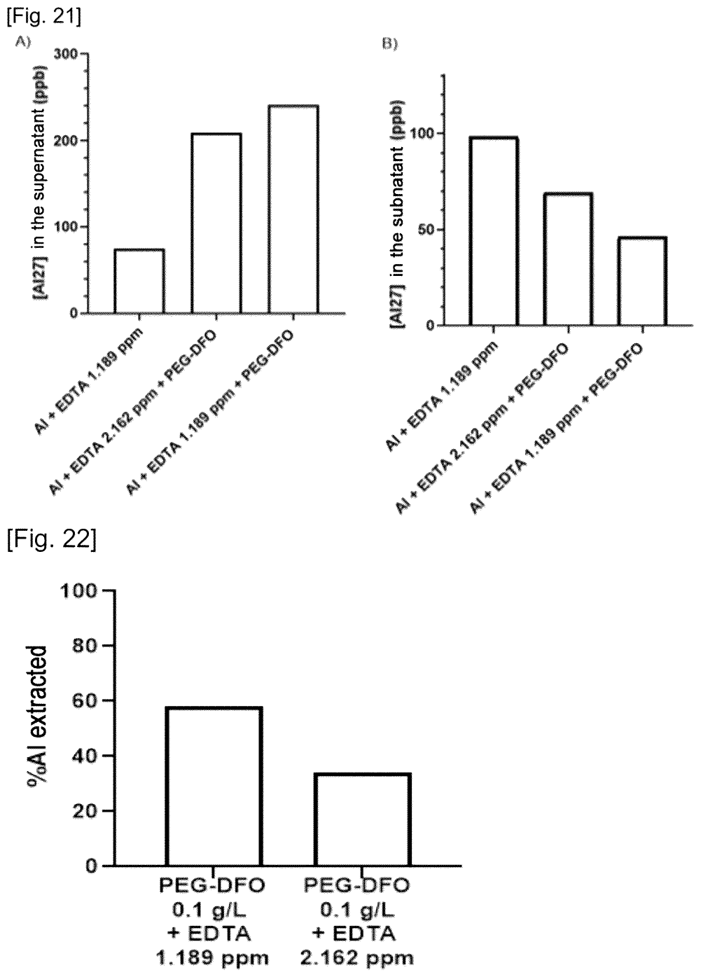

In the experiment, the polymer was added to a solution already containing aluminum and EDTA. After a 30 min incubation time, the samples were centrifuged in Vivaspin 20 tubes (4000 rpm, 10 min cycle). These tubes have a 30 kDa membrane, which allows the polymer to be retained above the membrane by virtue of its size greater than 30 kDa, while allowing smaller species such as EDTA and free aluminum to pass through. According to protocol 3, after each centrifugation cycle, the subnatant is recovered and the tube is topped up again to 20 mL with the initial solution, until the 100 mL of initial solution are centrifuged and 20 mL of sample are recovered in the supernatant. All the samples (initial solution, supernatant and subnatant) were then analyzed by ICP-MS to determine the aluminum concentration in each fraction (Table 18). For the two samples containing the polymer, the over-concentration of aluminum in the supernatant relative to the initial solution demonstrates the capture of aluminum by the polymer (Table 18 and FIG. 21). Furthermore, the concentration of aluminum in the subnatant is reduced in the presence of polymer, confirming the ability of PEG-DFO to capture aluminum at low concentration in the presence of EDTA (1 to 2 ppm) (FIG. 21). Specifically, in a pH 6 solution containing 100 ppb of aluminum and about 1 ppm of EDTA, 0.1 g/L of PEG-DFO can extract more than 50% of the aluminum (FIG. 22).

TABLE 18

| | | | PEG-DFO 0.1 g/L + | PEG-DFO 0.1 g/L + |
|---|---|---|---|---|
| | Al 100 ppb + | PEG-DFO | Al 100 ppb + | Al 100 ppb + |
| | EDTA 1.189 ppm | 0.1 g/L | EDTA 1.189 ppm | EDTA 2.162 ppm |
| Initial solution | 102.873 | 7.041 | 105.353 | 104.992 |
| Subnatant | 98.327 | 4.548 | 46.266 | 69.170 |
| Supernatant | 75.187 | 7.927 | 241.413 | 208.851 |

1. A dialysis fluid comprising a polymer with a weight-average molecular mass of between 20 kDa and 1000 kDa bearing at least one agent for chelating at least one metal such as lead, aluminum, chromium, nickel or cadmium, said polymer being present in a concentration of between 0.01 mg/L and 10 mg/L.

2. The dialysis fluid as claimed in claim 1, characterized in that the metal is chosen from heavy metals such as lead or cadmium.

3. The dialysis fluid as claimed in claim 1, characterized in that the chelating agent is chosen from the group consisting of DOTA, NOTA, NODAGA, DOTAGA, DOTAM, DO3AM, NOTAM, DOTP, NOTP, TETA, TETAM, DTPA, EDTA and DFO, preferably from the group consisting of DOTAGA, DFO, DOTAM and DTPA, more preferably DOTAGA.

4. The dialysis fluid as claimed in claim 1, characterized in that the polymer is a random polysaccharide bearing at least one chelating agent.

5. The dialysis fluid as claimed in claim 1, characterized in that the polymer is a random polysaccharide of formula I: 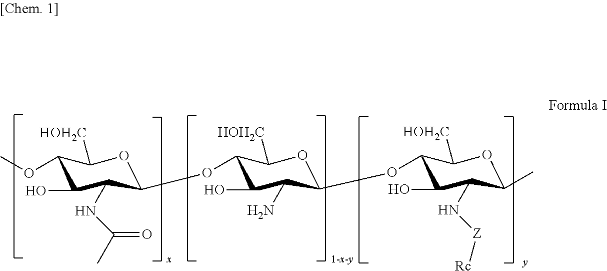
in which:
each Rc independently represents a group including a chelating agent,
each Z independently represents a linkage which may be a single bond or a hydrocarbon-based chain including between 1 and 12 carbon atoms, said chain possibly being linear or branched and possibly including one or more unsaturations and possibly including one or more heteroatoms, preferably chosen from nitrogen, oxygen, sulfur and atoms of the halogen family,
x is between 0.005 and 0.7, preferably between 0.05 and 0.7,
preferentially between 0.2 and 0.6, and more preferentially between 0.25 and 0.4,
y is between 0.01 and 0.7, preferably between 0.05 and 0.2,
the ratio y/x being greater than or equal to 0.05, preferably greater than or equal to 0.15, and
the sum x+y being greater than or equal to 0.30, preferably greater than or equal to 0.35.

6. The dialysis fluid as claimed in claim 1, characterized in that the polymer is a biocompatible polymer, for example of the polyethylene glycol (PEG) type.

7. A process for preparing a dialysis fluid as claimed in claim 1, characterized in that it comprises a step of diluting with water a concentrated solution or additive for a concentrated dialysis solution, preferably an acidic concentrated solution comprising, in addition to the polymer, a sufficient amount of acetic acid, hydrochloric acid or citric acid.

8. The process for preparing a dialysis fluid as claimed in claim 7, characterized in that the concentrated solution comprises one or more electrolytes, in particular chosen from sodium, potassium, chlorine, magnesium or calcium, and bicarbonates.

9. The process for preparing a dialysis fluid as claimed in claim 7, characterized in that the solution is concentrated and packaged in a bag suitable for a dialysis device, preferably a bag containing a solution volume of between 500 and 5000 mL, and an amount of said polymer of between 5 and 5000 mg, preferably between 10 and 100 mg.

10. A method for capturing at least one toxic metal such as lead, aluminum, chromium, nickel or cadmium, present in a dialysis fluid, comprising a step of adding to said dialysis fluid a sufficient amount of a polymer with a weight-average molecular mass of between 20 kDa and 1000 kDa bearing at least one agent for chelating at least one toxic metal such as lead, aluminum, chromium, nickel or cadmium, thus allowing capture of said toxic metal by chelation with the chelating agent of said polymer.

11. The capture method as claimed in claim 10, characterized in that the toxic metal is aluminum.

12. The capture method as claimed in claim 10, characterized in that the toxic metal is lead and the concentration in the dialysis fluid is less than 0.1 ppb.

13. The capture method as claimed in claim 10, characterized in that the toxic metal is cadmium and the concentration in the dialysis fluid is less than 0.01 ppb.

14. The capture method as claimed in claim 10, characterized in that at least 120 L of dialysis fluid is used in one patient dialysis.

15. The capture method as claimed in claim 10, characterized in that the dialysis fluid is introduced into a tank of a dialysis device, which can be connected to an extracorporeal blood circulation device comprising a porous dialysis membrane.

16. The method as claimed in claim 13, characterized in that the cutoff threshold of the porous membrane is less than the polymer size.

17. Use of a polymer with a weight-average molecular mass of between 20 kDa and 1000 kDa bearing at least one agent for chelating at least one toxic metal such as lead, aluminum, chromium, nickel or cadmium for preparing a dialysis composition, for example for hemodialysis, hemodiafiltration or hemofiltration, in particular a composition as claimed in claim 1.
